(19) United States
(12) Patent Application Publication
(10) Pub. No.: US 2006/0188898 A1
(43) Pub. Date: Aug. 24, 2006

(54) Markers for roscovitine

(75) Inventors: Simon R. Green, Dundee (GB); Paul Workman, Sutton (GB); Steven R. Whittaker, Sutton (GB)

Correspondence Address: PALMER & DODGE, LLP;KATHLEEN M. WILLIAMS, 111 HUNTINGTON AVENUE, BOSTON, MA, 02199, US

(73) Assignee: Cyclacel Limited

(21) Appl. No.: 11/238,251

(22) Filed: Sep. 29, 2005

Related U.S. Application Data
(63) Continuation of application No. PCT/GB04/01337, filed on Mar. 26, 2004.

(30) Foreign Application Priority Data
Apr. 2, 2003 (GB) 0307640.3

(51) Int. Cl. C12Q 1/68; A61K 31/522

(52) U.S. Cl. 435/6; 514/263.36

(57) ABSTRACT

The present invention relates to pharmacodynamic markers for CDKIs including the candidate 2,6,9-tri-substituted purine known as roscovitine. The identity of these markers facilitates the convenient identification of roscovitine-like activity both in vitro and in vivo.

The present invention relates to pharmacodynamic markers for cyclin dependant kinase inhibitors. In particular, the present invention relates to pharmacodynamic markers for the candidate 2,6,9-tri-substituted purine known as roscovitine (CYC 202) and roscovitine-like compounds. The identity of these markers facilitates the convenient identification of roscovitine-like activity both in vitro and in vivo.

A growing family of cyclin dependent kinase inhibitors (CDKI's) have been identified. These inhibitors have varying activities against the multiple CDK family members. Generally, these inhibitors bind to the ATP binding pockets of CDKs.

The 2,6,9-tri-substituted purines are becoming a well studied class of compound showing promise as CDKI's of use in the treatment of proliferative disorders such as cancers and leukemias. Fischer P & Lane D (Curr Med Chem (2000), vol 7, page 1213) provides a detailed review of CDKI's, their origins and described activities. In particular, roscovitine has been shown to inhibit CDK1, CDK2, CDK5, CDK7 and CDK9 and to block cell cycle progression in late G1/early S and in M-phase. The compound (R)-2-[(1-ethyl-2-hydroxyethyl)amino]-6-benzylamino-9-isopropylpurine, known as R-roscovitine was first described in WO97/20842 (Meijer L et al) and has since been developed as a promising candidate anti-cancer agent.

In the development of such agents, extensive pharmacokinetic and pharmacodynamic investigations must be undertaken in order to understand the actual mechanism of action upon administration and satisfy the regulatory authorities requirements as to toxicity and dosing. Such analysis is based upon the complex biochemistry of the cell cycle control system and detailed studies undertaken in the pre-clinical phase of drug development to ascertain the particular mode of activity of the candidate drug.

Of particular advantage in the pharmacokinetic and pharmacodynamic investigations is the identity of specific markers of activity for the candidate drug.

The present invention relates to the observation that a number of genes identified in any of FIGS. 1 through 8 act as specific pharmacodynamic (PD) markers for the activity of the cyclin dependant kinase inhibitor, roscovitine. In particular, the expression of the genes identified is up or down regulated after roscovitine treatment.

Thus, in a first aspect the invention relates to a method of monitoring the activity of a CDKI comprising:

(i) administering said CDKI to a cell, group of cells, an animal model or human; and

(ii) measuring gene expression in samples derived from the treated and the untreated cells, animal or human; and

(iii) detecting an increase or a decrease in gene expression of at least one of the genes identified in any of FIGS. 1 through 8 in the treated sample as compared to the untreated sample as an indication of CDKI activity.

Preferably, the CDKI is a compound having roscovitine activity. Most preferably, the CDKI is roscovitine or a roscovitine analogue or derivative.

Detection of gene expression may be performed by any one of the methods known in the art, particularly by microarray analysis, Western blotting or by PCR techniques such as QPCR.

Suitably, a number of the biomarkers of roscovitine activity (i.e. genes identified in any of FIGS. 1 through 8) may be observed in combination.

Preferably, where roscovitine is administered to a human, the effective concentration of roscovitine administered to a cell is greater than 5 micromolar and, more preferably greater than 10 micromolar.

Suitably, where roscovitine is administered to a human, treatment with the drug is for 2, 4 or 8 hours prior to removing blood samples for analysis of gene expression.

In one embodiment, where roscovitine is administered to a cell, the effective concentration of roscovitine is preferably upto 75 micromolar.

In one embodiment, the cell, group of cells, animal model or human, is treated with roscovitine at 7.5, 15 or 30 micromolar for 1.5 hours before analysis to detect gene expression. In another embodiment, the cell, group of cells, animal model or human, is treated with roscovitine at 7.5, 15 or 30 micromolar for 3 hours before analysis to detect gene expression. In a further embodiment, the cell, group of cells, animal model or human, is treated with roscovitine at 15, 45 or 75 micromolar for 2 hours before analysis to detect gene expression. In a yet further embodiment, the cell, group of cells, animal model or human, is treated with roscovitine at 15, 45 or 75 micromolar for 4 hours before analysis to detect gene expression.

Preferably, the cell, group of cells, animal model or human, is treated with roscovitine at 50 micromolar for 4, 12, 24 or 48 hours before analysis to detect gene expression. In this embodiment, a change in gene expression of at least one of the genes identified in any of FIGS. 1 through 8 is detected as an indication of roscovitine activity. In this embodiment, gene expression in cells is preferably detected in cells having a phenotype similar to HT29.

In another embodiment, a decrease in any one of the genes identified in any of FIGS. 1, 3, 5 and 7 or an increase in any one of the genes identified in FIGS. 2, 4, 6, and 8 is identified.

used herein the terms “roscovitine” and “R-roscovitine” are used to refer to the compound 2-(R)-(1-ethyl-2-hydroxyethylamino)-6-benzylamino-9-isopropylpurine, also referred to as CYC202. In its unqualified form the term “roscovitine” is used to include the R-roscovitine, the S enantiomer and racemic mixtures thereof. This compound and its preparation are described in U.S. Pat. No. 6,316,456. Analogues of roscovitine are described, for example, in WO 03/002565.

In a preferred embodiment of the invention roscovitine is administered to a mammal or a human, more preferably a human. When performed on an animal model, the invention is preferably performed on a tumour model such as a xenograft mouse model comprising a tumour cell line such as HT29 or A549.

Suitably changes in gene expression are monitored in samples taken from the mammal or human. Suitable samples include tissue samples such as biopsy, blood, urine, buccal scrapes etc. In one embodiment, expression is preferably detected in tumour cells, particularly cells derived from a tumour such as breast, lung, gastric, head and neck, colorectal, renal, pancreatic, uterine, hepatic, bladder, endometrial and prostate cancers and leukemias or from blood cells such as lymphocytes and, preferably, peripheral lymphocytes such as PBMC.

As used herein, the term “PBMC” refers to peripheral blood mononuclear cells and includes PBLs (peripheral blood leucocytes).

When the invention is performed ex vivo, it is preferably performed on a group of cells, preferably a cell culture. Preferred cell types are selected from colonic tumour cell lines such as HT29, lung tumour cell lines such as A549, renal tumour cell lines such as A498, bladder tumour cell lines such as HT13, breast tumour cell lines such as MCF7, endometrial tumour cell lines such as AN3CA, uterine tumour cell lines such as MESSA DH6 uterine sarcoma cells, hepatic tumour cell lines such as Hep2G, prostate tumour cell lines such as DU145, T cell tumour cell lines such as Cem T cell, pancreatic tumour cell lines such as MiaPaCa2. Alternatively, the cells may be in the form of a histological sample of a tumor biopsy. As such, the invention further relates to a method of detecting a proliferative cell in a sample comprising a method as described above. In another alternative, the cells may be blood cell cultures such as PBMCs.

The methods of the present invention where the levels of expression of any of the genes identified herein are monitored will preferably involve monitoring the levels prior to administration of roscovitine and then again preferably 1.5, 2, 3, 4, 5, 8, 12, 24 or 48 hours after administration. In a preferred embodiment, the level is monitored again at least 1.5 hours after administration of roscovitine.

In one preferred embodiment, the level of a gene detected after administration of roscovitine is preferably lower than that detected prior to administration of roscovitine.

In another preferred embodiment, where the gene whose expression is detected is one of the genes identified in FIGS. 2, 4, 6, and 8, the level of a gene detected after administration of roscovitine is preferably higher than that detected prior to administration of roscovitine.

The second aspect of the invention relates to the independent monitoring of roscovitine activity by monitoring the levels of gene expression. In a preferred embodiment, this monitoring is conducted together with the monitoring of gene expression. In one embodiment, the level of gene expression detected after administration of roscovitine is preferably higher than that detected prior to administration of roscovitine. In another embodiment, the level of gene expression detected after administration of roscovitine is preferably lower than that detected prior to administration of roscovitine.

The methods of the present invention may be further utilised in;

(a) methods of assessing suitable dose levels of roscovitine comprising monitoring the degree and rate of gene expression after administration of roscovitine to a cell, group of cells, animal model or human,

(b) methods of identifying a candidate drug having roscovitine-like activity comprising administering said candidate drug to a cell, group of cells, animal model or human and monitoring the presence or absence of a gene or

Methods such as described in (a) may further comprise correlating the degree and rate of gene expression with the known rate of inhibition of a known gene whose expression is modulated by roscovitine at the same dosage, over the same time period. In one embodiment, phosphorylation status of RB may be compared to the pattern of expression of any one of the genes identified herein. RB as a marker of roscovitine activity is described in WO 02/061386.

In a further aspect, the invention relates to the use of a gene in the monitoring of activity of roscovitine utilising any of the methods described above.

In an even further aspect, the invention relates to kits for assessing the activity of roscovitine. Suitably kits may comprise probes for detecting gene expression of at least one of the genes identified herein or antibodies which bind to the protein product of at least one of the genes identified herein.

For example, suitable kits may be kits for QPCR analysis comprising primers for the detection of expression of at least one of the genes identified herein. Suitably, kits for QPCR analysis may detect at least one gene, and may also comprise primers directed to another gene identified herein.

Other such kits may preferably comprise the antibodies recognising the protein product of a gene identified herein alone or in combination With antibodies directed to another gene identified herein.

Suitable cell lines for the pharmacodynamic investigation of roscovitine and related compounds include colonic tumour cell lines such as HT29, lung tumour cell lines such as A549, renal tumour cell lines such as A498, bladder tumour cell lines such as HT13, breast tumour cell lines such as MCF7, endometrial tumour cell lines such as AN3CA, uterine tumour cell lines such as MESSA DH6 uterine sarcoma cells, hepatic tumour cell lines such as Hep2G, prostate tumour cell lines such as DU145, T cell tumour cell lines such as Cem T cell, pancreatic tumour cell lines such as MiaPaCa2, and suitable animal models include xenograft mouse models lines such as HT29 and A549 xenograft mouse models (cell lines & models available from ATCC). Antibodies for genes may be derived from commercial sources or through techniques which are familiar to those skilled in the art.

Typically in cell line investigations a CDK2 inhibitory (IC₅₀) dosage of roscovitine is administered and samples extracted over a 24 or 48 hour time period for example at 2, 4, 12, 24 and 48 hours after administration. Protein samples are isolated, loaded and resolved on SDS-PAGE, blotted and probed for the appropriate marker. When conducting investigation in animal models or humans, a suitable proliferating tissue must be identified as being a source of cells that can be extracted from the animal or human for assessment of roscovitine activity. Suitable tissue includes any proliferating tissue. In particular including a tumor biopsy, but it has now been observed that circulating lymphocytes and cells of the buccal mucosa may also be used. Once extracted, these cells can be treated in a manner identical to that described for cell lines. In most cases a pool of markers including a gene as identified herein.

Suitable methods for detecting gene expression in biopsy samples include using FISH or immunohistochemistry techniques using antibodies that recognise the genes identified herein.

This embodiment of the invention may be further developed to use the effect of roscovitine on gene expression as a tool in dose titration i.e. by monitoring the degree and rate of gene expression a suitable dose of roscovitine may be determined. Such analysis may further involve correlation of changes of gene expression with the known rate of inhibition of, for example, either CDK2 or RB phosphorylation by roscovitine at the same dosage. In this manner, a single measurement of the rate and degree of gene expression may be taken as indicative of further activities of roscovitine.

In an even further embodiment of the invention the gene expression level by a candidate drug may be taken as an indication of its mode of activity in that it may be classified as roscovitine-like.

In accordance with either the first or second aspects, the present invention further relates to a kit for assessing the activity of roscovitine comprising nucleic acid primers or antibodies for at least one of the genes as identified herein. Preferably, the kit comprises nucleic acid primers or antibodies for any one of the genes as identified herein alone or in combination with another gene as identified herein. The kits may be used in accordance with any of the hereinbefore described methods for monitoring roscovitine activity, assessing roscovitine dosage or the roscovitine-like activity of a candidate drug.

Response of a cancer patient to treatment with a particular course of therapy can be highly variable. For example, a patient may be sensitive to treatment with a particular therapy and therefore exhibit reduced tumour burden or improved symptoms. Alternatively, a patient may be resistant to treatment and show no or little improvement in response to a particular therapy. Detecting genes whose expression is modified by a CDKI such as roscovitine may also be useful in methods of identifying markers for the prediction of a response to treatment with a CDKI.

Accordingly, in another aspect there is provided a method for identifying genes whose expression in tumours enables a response to treatment with a CDKI such as roscovitine to be predicted, said method comprising:

a) taking a sample from a patient showing sensitivity to treatment with a CDKI such as roscovitine and detecting expression of at least one of the genes as identified herein;

b) taking a sample from a patient showing resistance to treatment with a CDKI such as roscovitine and detecting expression of at least one of the genes as identified herein; and

c) comparing the patterns of gene expression from a) and b) and therefore identifying those genes which correlate with sensitivity and those which correlate with resistance.

Patterns of gene expression from tumours may then be determined and a particular tumour classified as “sensitive” or “resistant” to treatment according to the expression of those marker genes identified according to the above method.

### BRIEF DESCRIPTION OF THE FIGURES

FIGS. 1A through 1E shows mRNA expression profiles for mRNAs having a normalised ratio of medians less than 0.5 in HT29 cells treated with 50 micromolar CYC202 for 4 hours.

FIGS. 2A through 2D shows mRNA expression profiles for mRNAs having a normalised ratio of medians greater than 2 in HT29 cells treated with 50 micromolar CYC202 for 4 hours.

FIGS. 3A through 3G shows mRNA expression profiles for mRNAs having a normalised ratio of medians less than 0.5 in HT29 cells treated with 50 micromolar CYC202 for 12 hours.

FIGS. 4A through 4G shows mRNA expression profiles for mRNAs having a normalised ratio of medians greater than 2 in HT29 cells treated with 50 micromolar CYC202 for 12 hours.

FIGS. 5A through 5K shows mRNA expression profiles for mRNAs having a normalised ratio of medians less than 0.5 in HT29 cells treated with 50 micromolar CYC202 for 24 hours.

FIGS. 6A through 6E shows mRNA expression profiles for mRNAs having a normalised ratio of medians greater than 2 in HT29 cells treated with 50 micromolar CYC202 for 24 hours.

FIGS. 7A through 7G shows mRNA expression profiles for mRNAs having a normalised ratio of medians less than 0.5 in HT29 cells treated with 50 micromolar CYC202 for 48 hours.

FIGS. 8A through 8F shows mRNA expression profiles for mRNAs having a normalised ratio of medians greater than 2 in HT29 cells treated with 50 micromolar CYC202 for 48 hours.

FIGS. 9A and 9B shows mRNA expression as assessed by microarray analysis and protein expression as assessed by Western Blot, respectively.

### DETAILED DESCRIPTION OF THE INVENTION

The practice of the present invention will employ, unless otherwise indicated, conventional techniques of chemistry, molecular biology, cell biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, IRL Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

By “roscovitine activity” or “roscovitine-like activity” is meant an activity exhibited by roscovitine. For example, roscovitine-like means capable of inhibiting cell cycle progression in late G1/early S or M phase. Preferably, said inhibition of cell cycle progression is through inhibiting CDKs including CDK1, CDK2, CDK5, CDK7 and CDK9. A study of roscovitine activity is reported in McClue et al. Int. J. Cancer, 2002, 102, 463-468.

The term “marker” or “biomarker” of roscovitine activity is used herein to refer to a gene whose expression in a sample derived from a cell or mammal is modulated, for example, up or down regulated, in response to treatment with roscovitine.

A sample derived from a treated or untreated cell can be a lysate, extract or nucleic acid sample derived from a group of cells which can be from tissue culture or animal or human. A cell can be isolated from an individual (e.g. from a blood sample) or can be part of a tissue sample such as a biopsy.

The term “expression” refers to the transcription of a gene's DNA template to produce the corresponding mRNA and translation of this mRNA to produce the corresponding gene product (i.e., a peptide, polypeptide, or protein).

By “polynucleotide” or “polypeptide” is meant the DNA and protein sequences disclosed herein whose expression is modified in response to roscovitine. The terms also include close variants of those sequences, where the variant possesses the same biological activity as the reference sequence. Such variant sequences include “alleles” (variant sequences found at the same genetic locus in the same or closely-related species), “homologs” (a gene related to a second gene by descent from a common ancestral DNA sequence, and separated by either speciation (“ortholog”) or genetic duplication (“paralog”)), so long as such variants retain the same biological activity as the reference sequence(s) disclosed herein.

The invention is also intended to include detection of genes having silent polymorphisms and conservative substitutions in the polynucleotides and polypeptides disclosed herein, so long as such variants retain the same biological activity as the reference sequence(s) as disclosed herein.

Measuring Expression of Gene Markers of Roscovitine Activity

Levels of gene expression may be determined using a number of different techniques.

a) at the RNA Level

Gene expression can be detected at the RNA level. RNA may be extracted from cells using RNA extraction techniques including, for example, using acid phenol/guanidine isothiocyanate extraction (RNAzol B; Biogenesis), or RNeasy RNA preparation kits (Qiagen). Typical assay formats utilising ribonucleic acid hybridisation include nuclear run-on assays, RT-PCR, RNase protection assays (Melton et al., Nuc. Acids Res. 12:7035), Northern blotting and In Situ hybridization.

For Northern blotting, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

Nuclease Protection Assays (including both ribonuclease protection assays and S1 nuclease assays) provide an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. NPAs allow the simultaneous detection of several RNA species.

In situ hybridization (ISH) is a powerful and versatile tool for the localization of specific mRNAs in cells or tissues. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with calorimetric or fluorescent reagents. This latter method of detection is the basis for Fluorescent In Situ Hybridisation (FISH).

Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection. Relative quantitative RT-PCR involves amplifying an internal control simultaneously with the gene of interest. The internal control is used to normalize the samples. Once normalized, direct comparisons of relative abundance of a specific mRNA can be made across the samples. Commonly used internal controls include, for example, GAPDH, HPRT, actin and cyclophilin.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U. et al., Science 242:229-237 (1988) and Lewis, R., Genetic Engineering News 10: 1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Qβ Replicase technique, RNA replicase for the bacteriophage Qβ, which replicates single-stranded RNA, is used to amplify the target DNA, as described by Lizardi et al., 1988, Bio/Technology 6:1197.

Quantitative PCR (Q-PCR) is a technique which allows relative amounts of transcripts within a sample to be determined.

Alternative amplification technology can be exploited in the present invention. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

Suitable probes for detected the markers of roscovitine activity identified herein may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like. Suitable kits may comprise, for example, primers for a QPCR reaction or labelled probes for performing FISH.

b) at the Polypeptide Level

Gene expression may also be detected by measuring the polypeptides encoded by the gene markers of roscovitine activity. This may be achieved by using molecules which bind to the polypeptides encoded by any one of the genes identified herein as a marker of roscovitine activity. Suitable molecules/agents which bind either directly or indirectly to the polypeptides in order to detect the presence of the protein include naturally occurring molecules such as peptides and proteins, for example antibodies, or they may be synthetic molecules.

Methods for production of antibodies are known by those skilled in the art. If polyclonal antibodies are desired, a selected mammal (e.g., mouse, rabbit, goat, horse, etc.) is immunised with an immunogenic polypeptide bearing an epitope(s) from a polypeptide. Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order to generate a larger immunogenic response, polypeptides or fragments thereof maybe haptenised to another polypeptide for use as immunogens in animals or humans.

Monoclonal antibodies directed against epitopes in polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides of the invention can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Standard laboratory techniques such as immunoblotting as described above can be used to detect altered levels of markers of roscovitine activity, as compared with untreated cells in the same cell population.

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Antibodies may be used in detecting markers of roscovitine activity identified herein in biological samples by a method which comprises: (a) providing an antibody of the invention; (b) incubating a biological sample with said antibody under conditions which allow for the formation of an antibody-antigen complex; and (c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues. Other suitable examples include blood or urine samples.

Antibodies that specifically bind to protein markers of roscovitine activity can be used in diagnostic methods and kits that are well known to those of ordinary skill in the art to detect or quantify the markers of roscovitine activity proteins in a body fluid or tissue. Results from these tests can be used to diagnose or predict the occurrence or recurrence of a cancer and other cell cycle progression-mediated diseases or to assess the effectiveness of drug dosage and treatment.

Antibodies can be assayed for immunospecific binding by any method known in the art. The immunoassays which can be used include but are not limited to competitive and non-competitive assay systems using techniques such as western blots, immunohistochemistry, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

Antibodies for use in the invention may be bound to a solid support and/or packaged into kits in a suitable container along with suitable reagents, controls, instructions and the like.

Arrays

Array technology and the various techniques and applications associated with it is described generally in numerous textbooks and documents. These include Lemieux et al., 1998, Molecular Breeding 4:277-289; Schena and Davis. Parallel Analysis with Biological Chips. in PCR Methods Manual (eds. M. Innis, D. Gelfand, J. Sninsky); Schena and Davis, 1999, Genes, Genomes and Chips. In DNA Microarrays: A Practical Approach (ed. M. Schena), Oxford University Press, Oxford, UK, 1999); The Chipping Forecast (Nature Genetics special issue; January 1999 Supplement); Mark Schena (Ed.), Microarray Biochip Technology, (Eaton Publishing Company); Cortes, 2000, The Scientist 14(17):25; Gwynne and Page, Microarray analysis: the next revolution in molecular biology, Science, 1999, Aug. 6; Eakins and Chu, 1999, Trends in Biotechnology, 17:217-218, and also at various world wide web sites.

Array technology overcomes the disadvantages with traditional methods in molecular biology, which generally work on a “one gene in one experiment” basis, resulting in low throughput and the inability to appreciate the “whole picture” of gene function. Currently, the major applications for array technology include the identification of sequence (gene/gene mutation) and the determination of expression level (abundance) of genes. Gene expression profiling may make use of array technology, optionally in combination with proteomics techniques (Celis et al., 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf et al et al., 2000, Nat Genet 24(3):236-44; Ross et al., 2000, Nat Genet 2000, 24(3):227-35), SNP analysis (Wang et al., 1998, Science 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica 30(2):155-77; Afshari et al., 1999, Cancer Res 59(19):4759-60) and toxicogenomics (a hybrid of functional genomics and molecular toxicology). The goal of toxicogenomics is to find correlations between toxic responses to toxicants and changes in the genetic profiles of the objects exposed to such toxicants (Nuwaysir et al., 1999, Molecular Carcinogenesis 24:153-159).

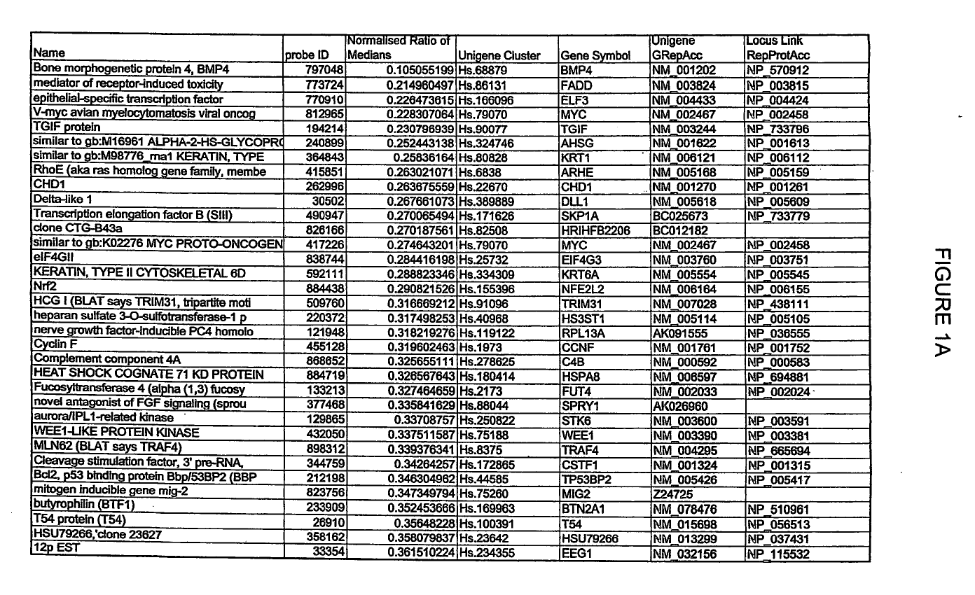

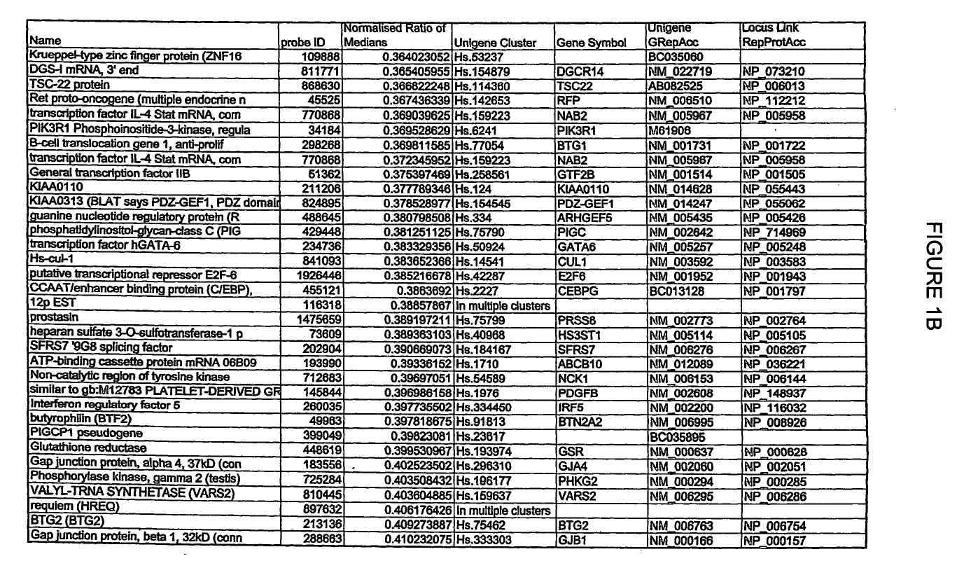

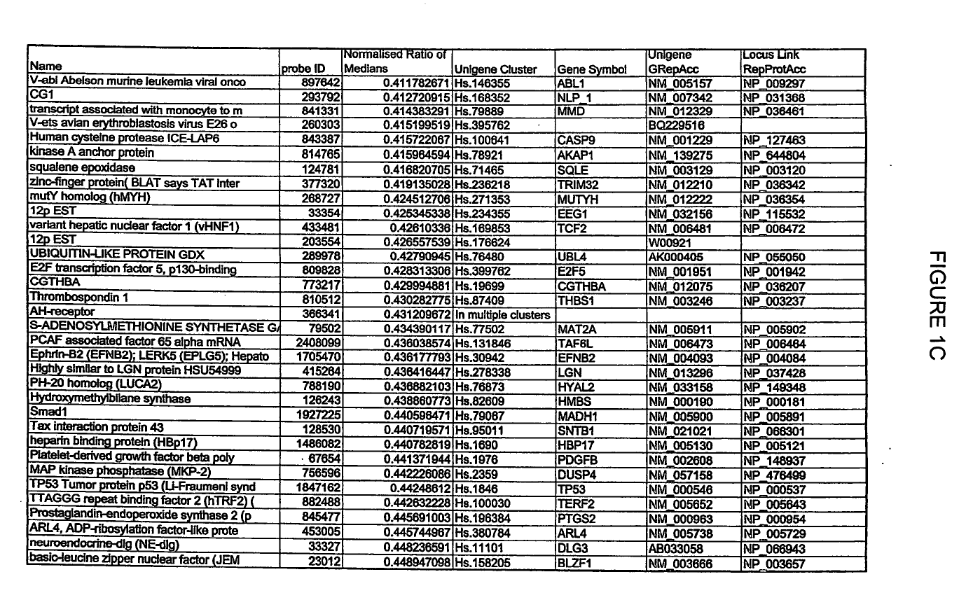

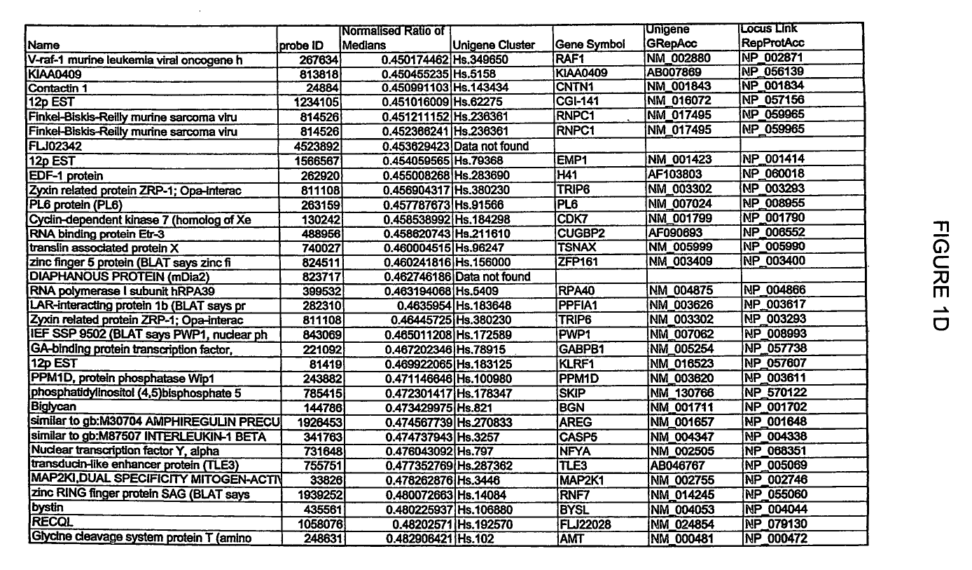

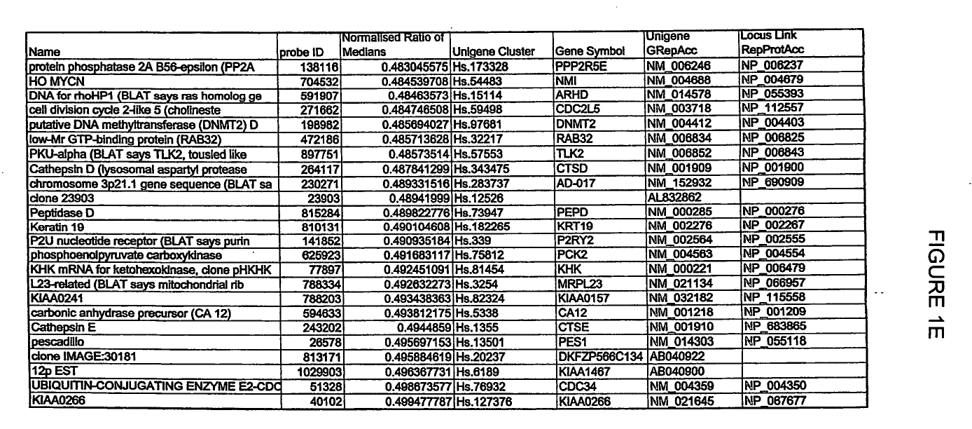

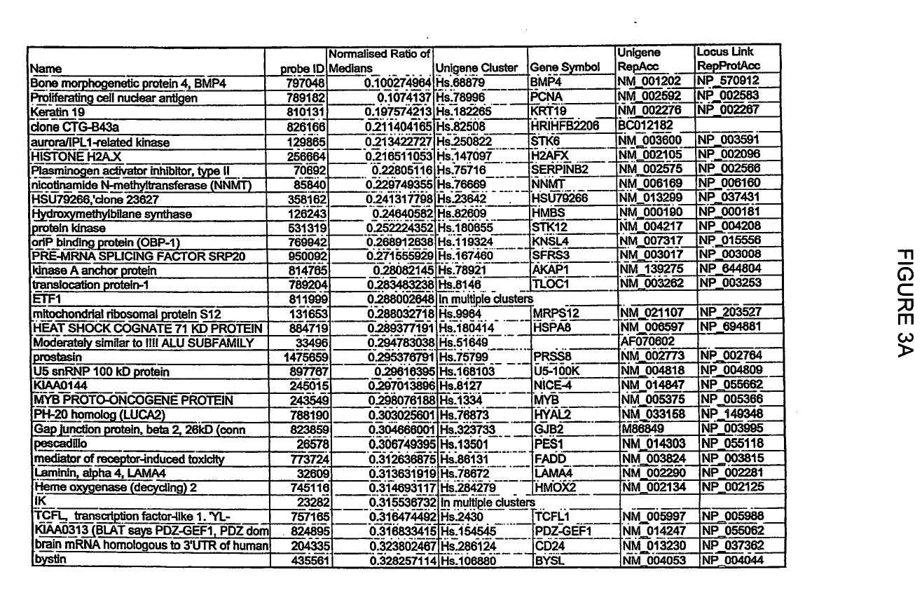

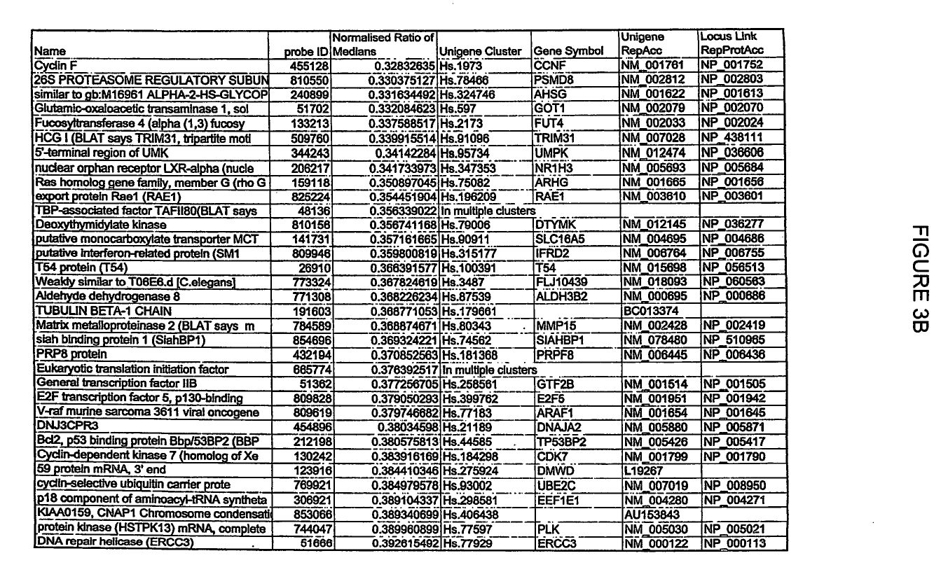

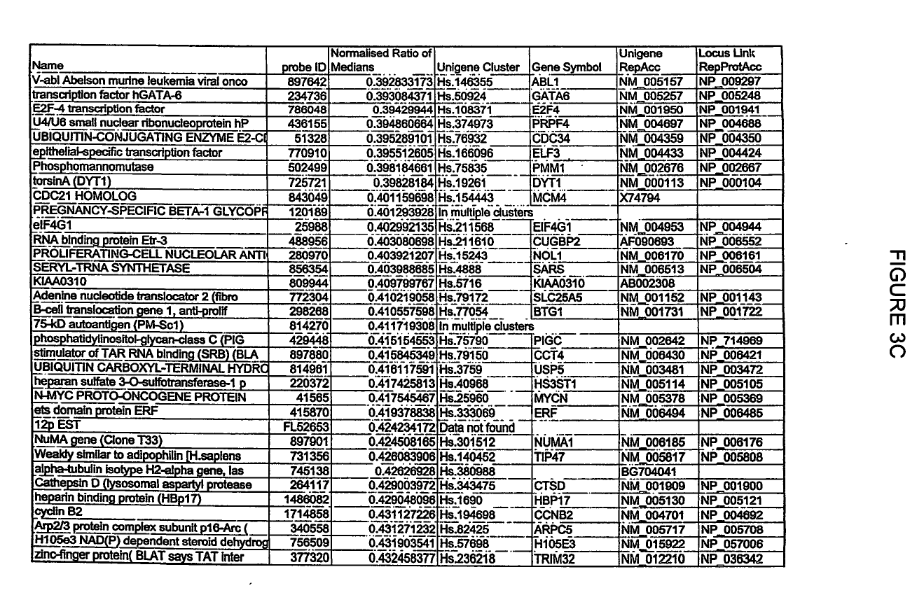

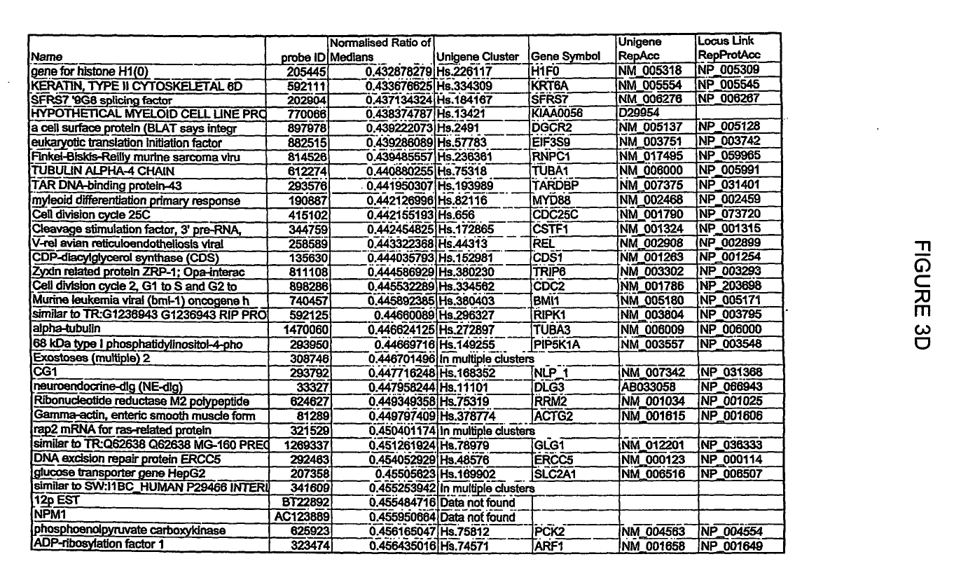

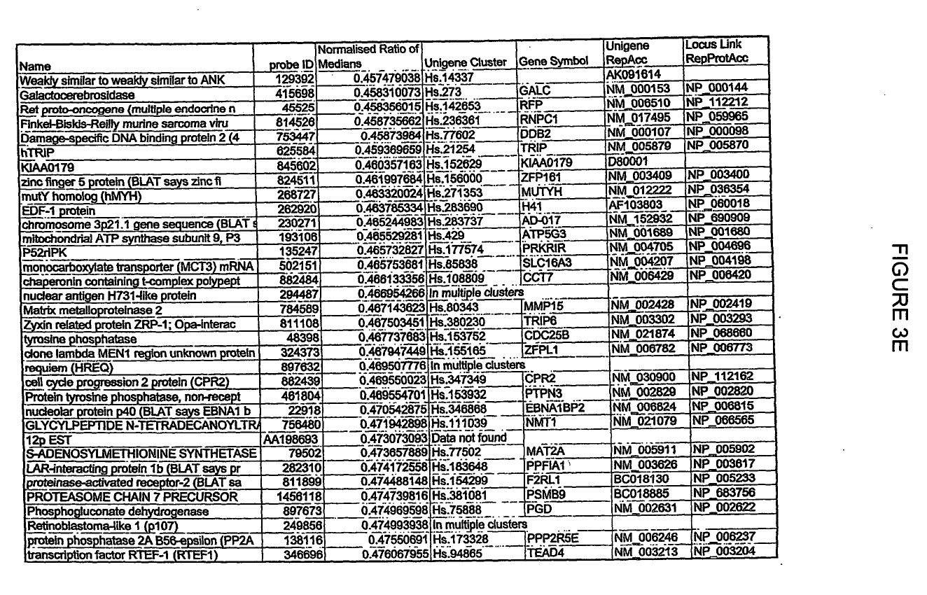

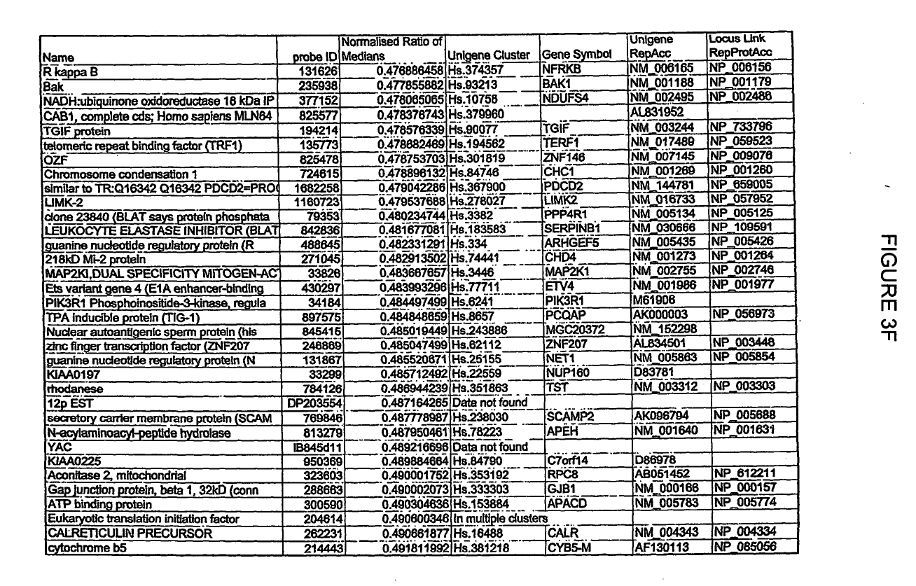

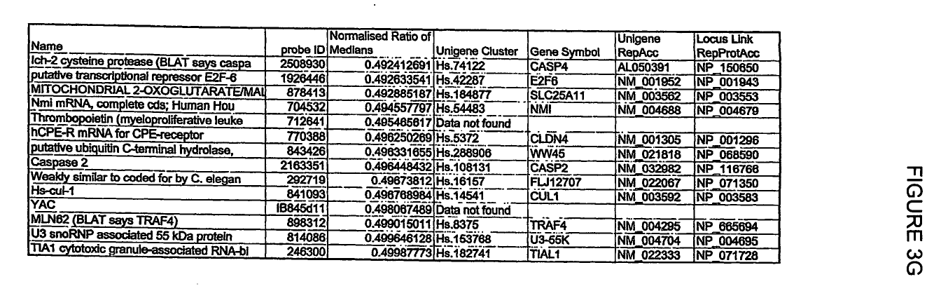

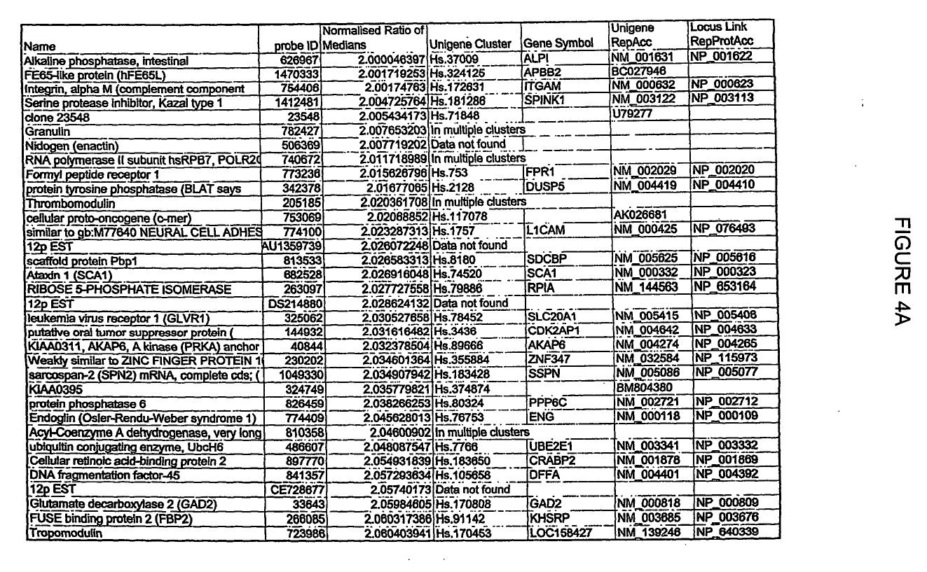

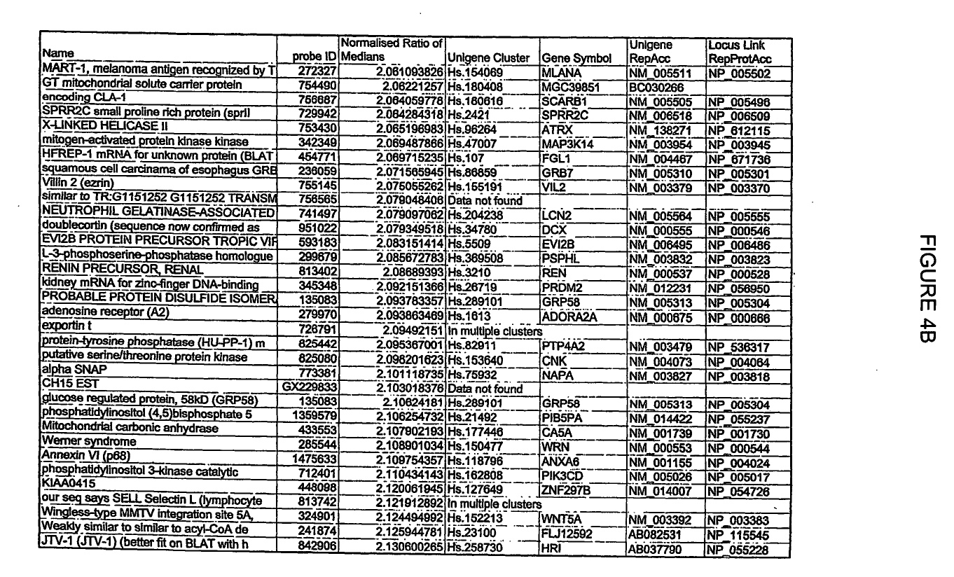

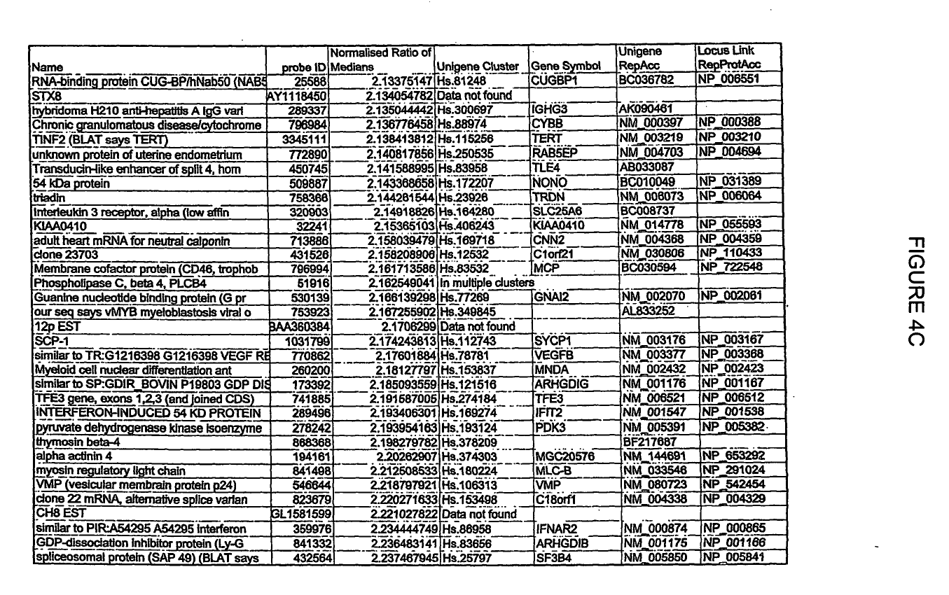

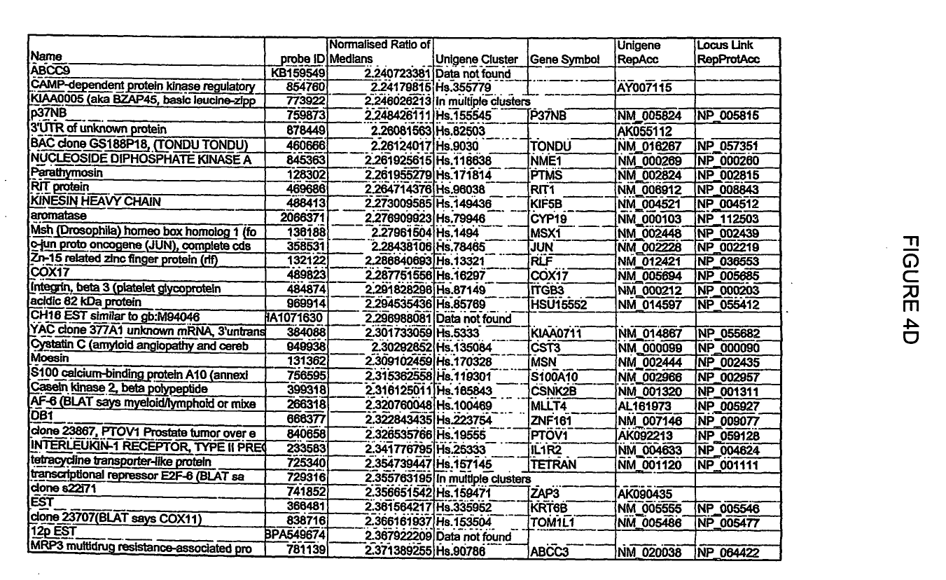

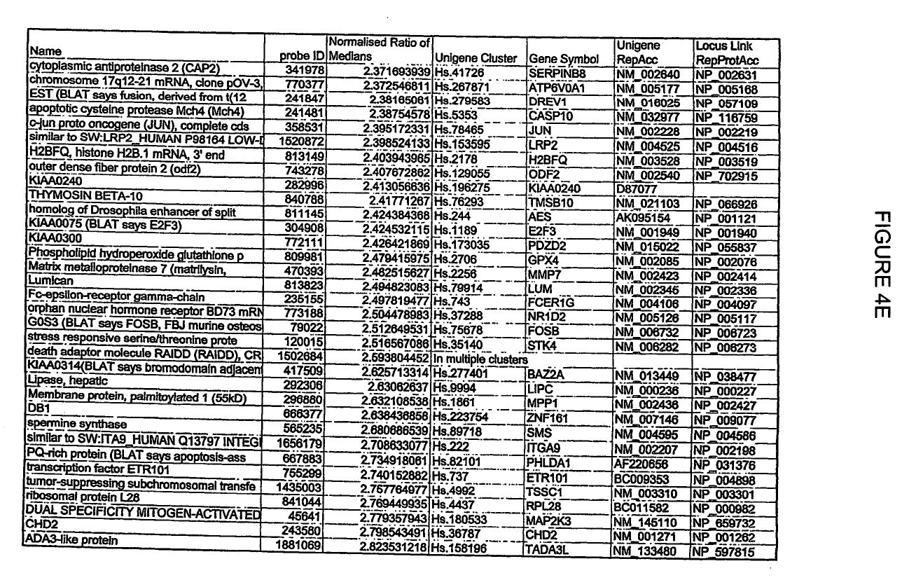

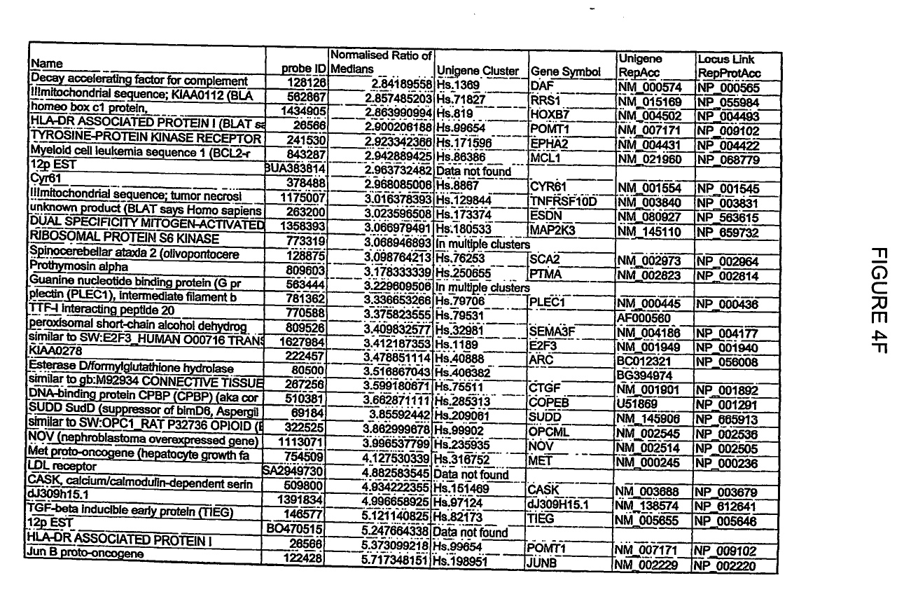

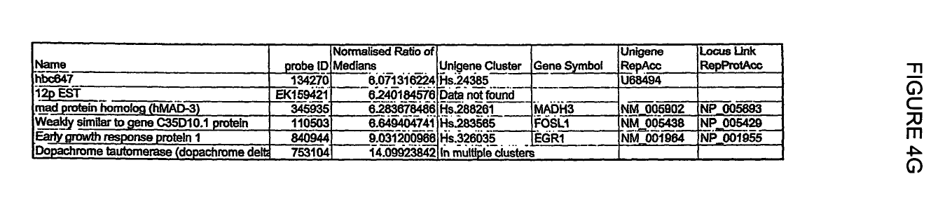

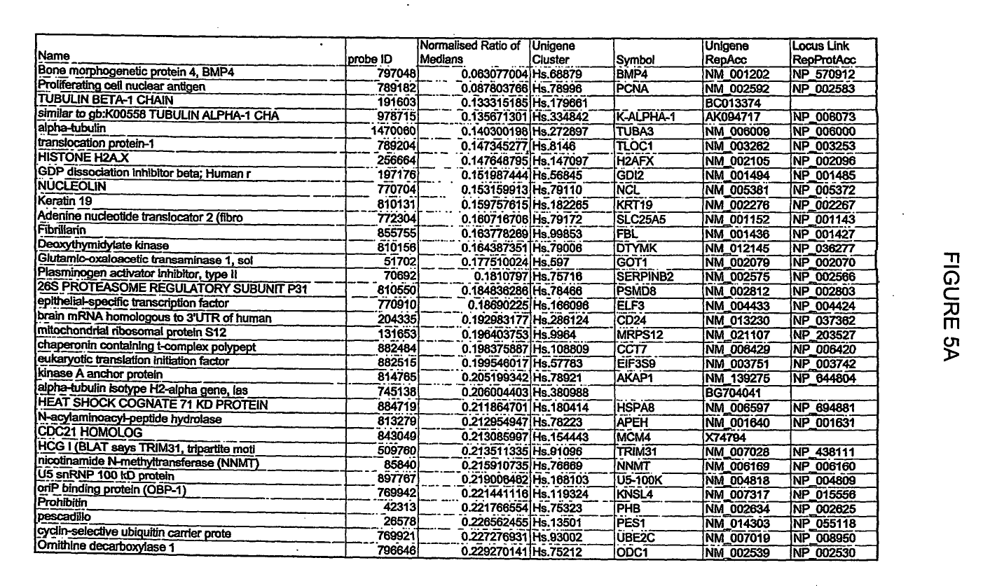

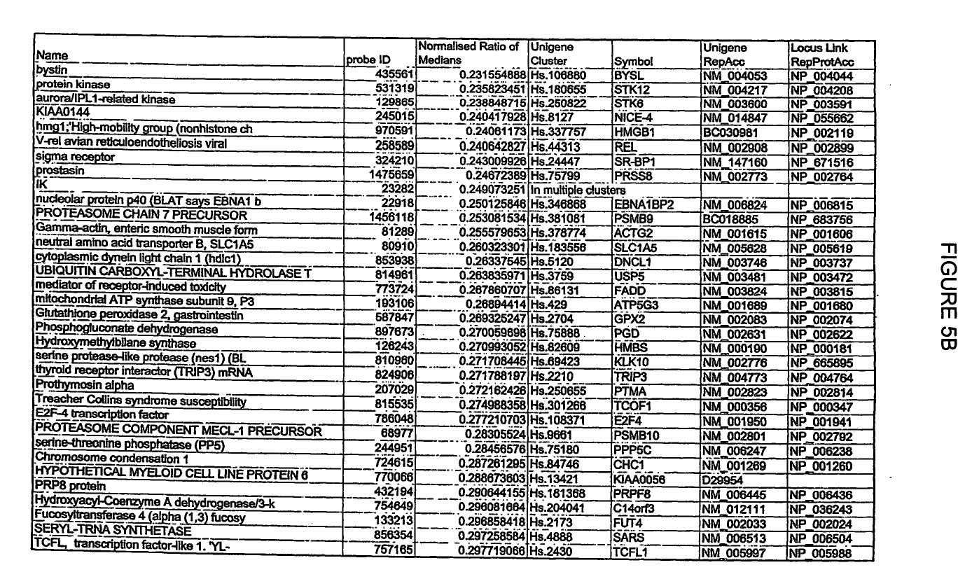

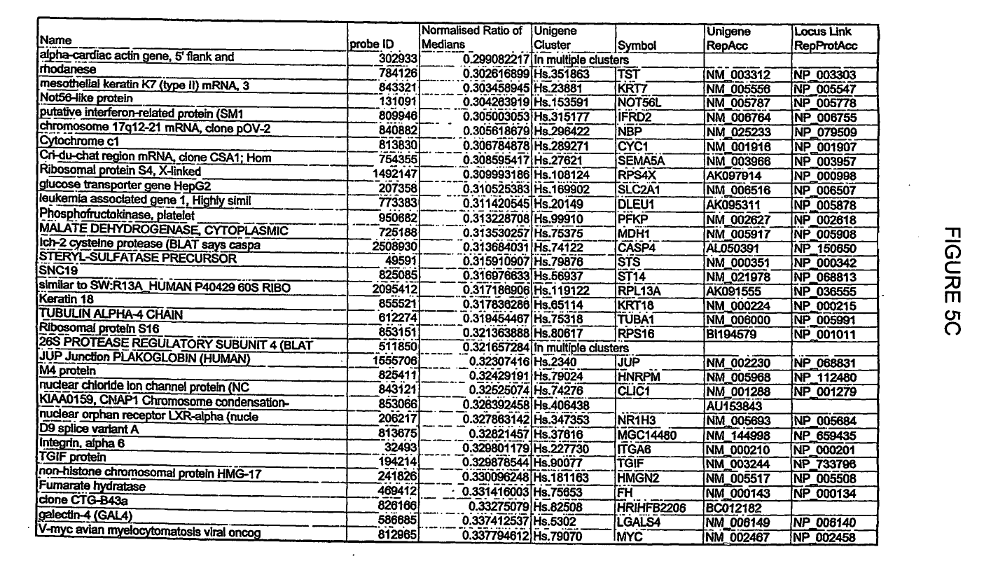

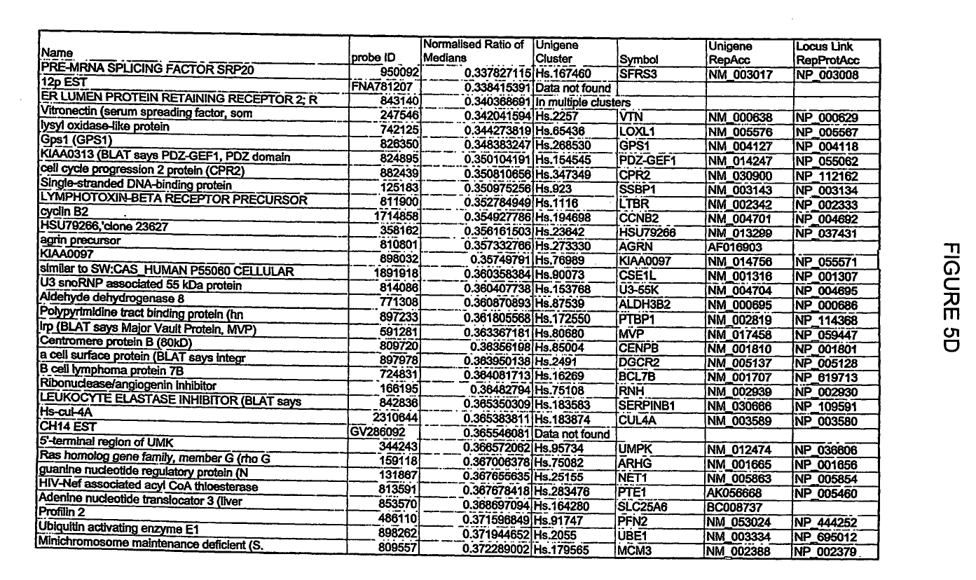

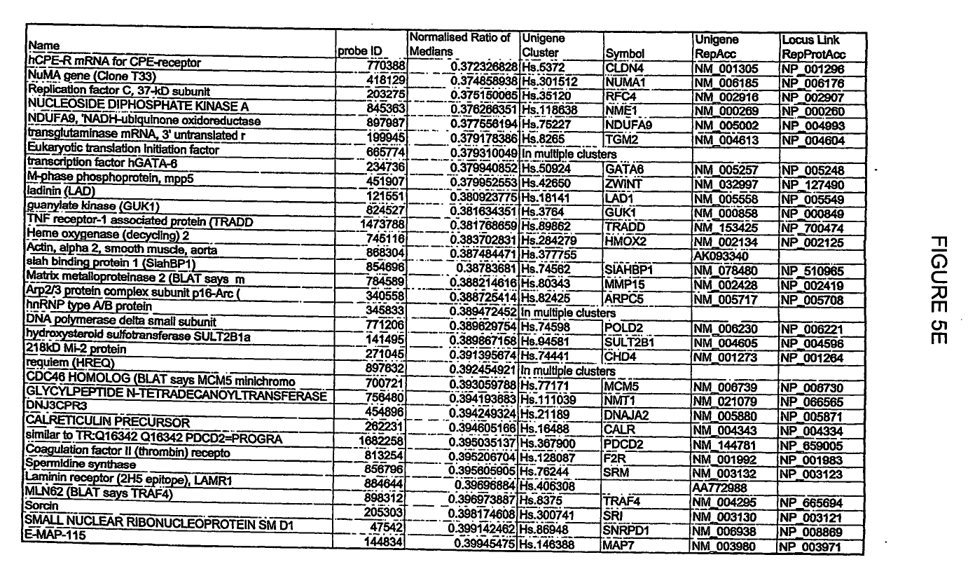

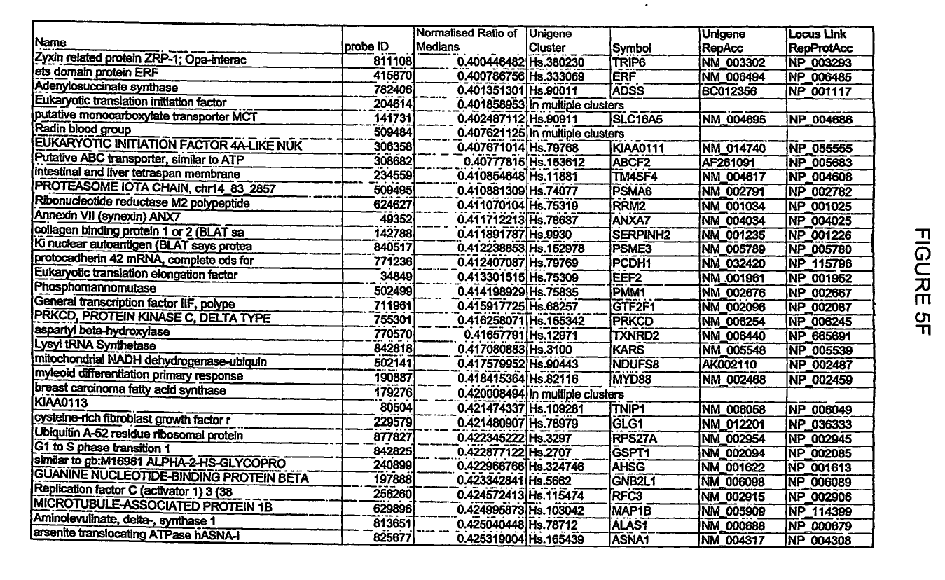

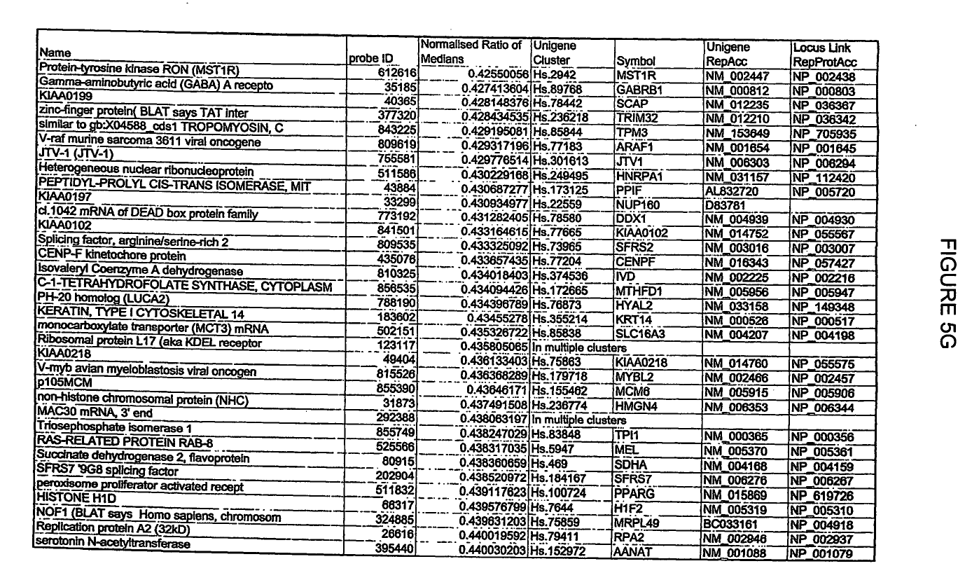

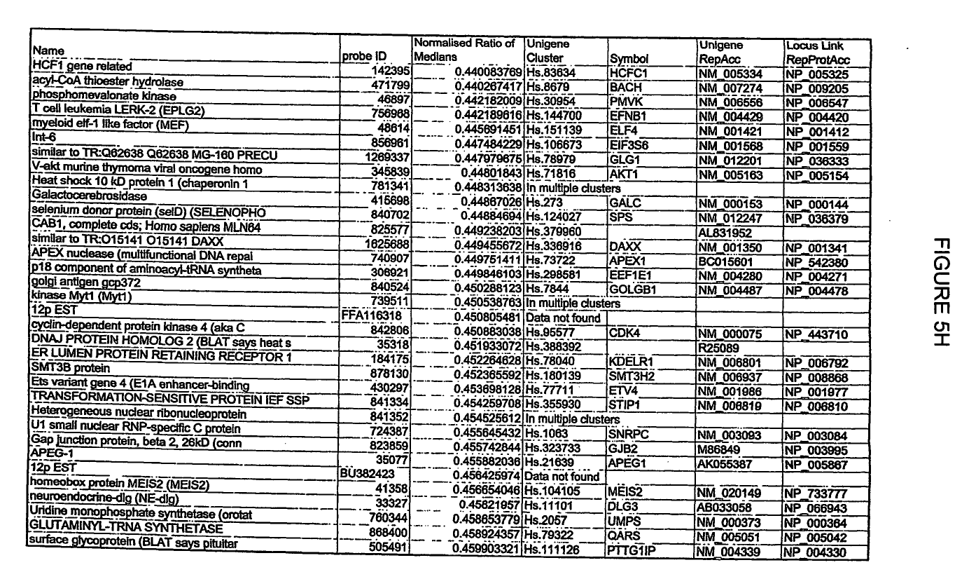

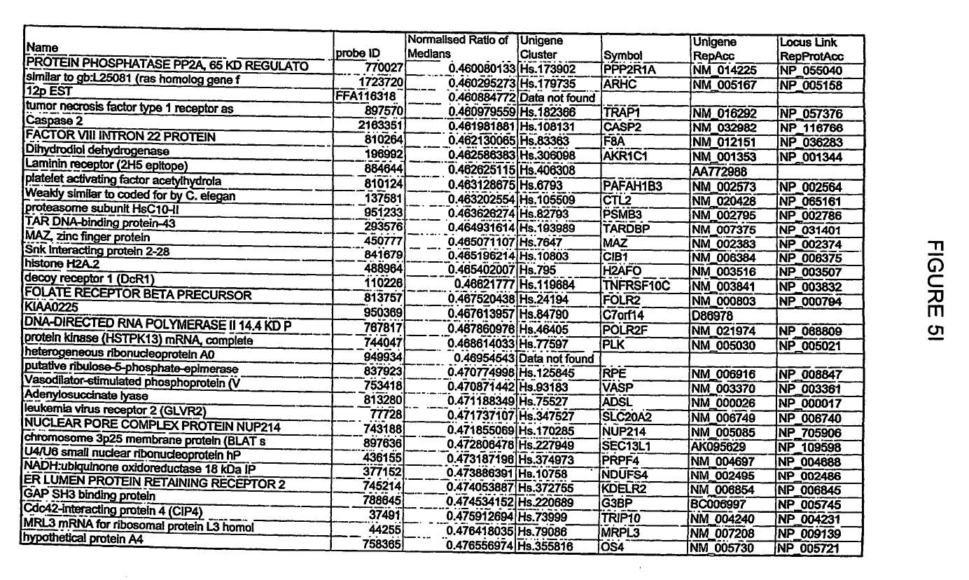

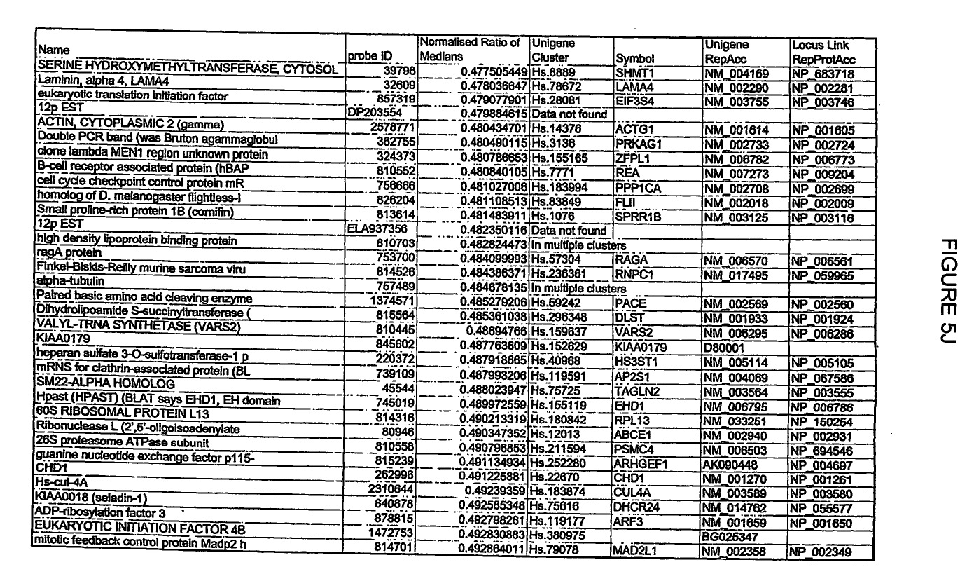

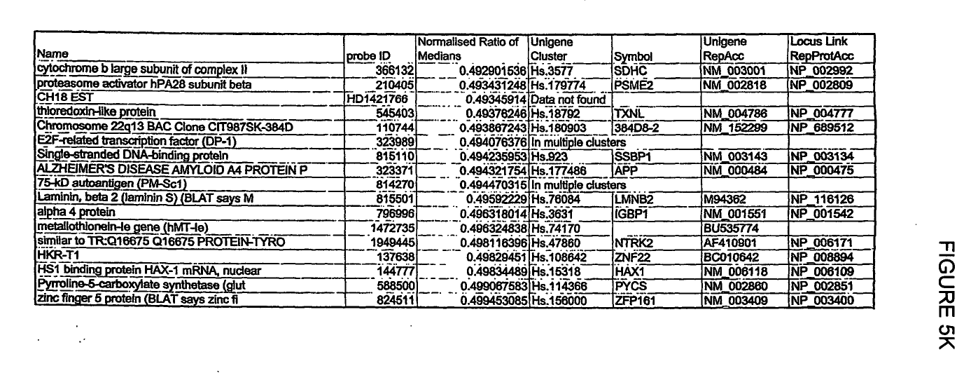

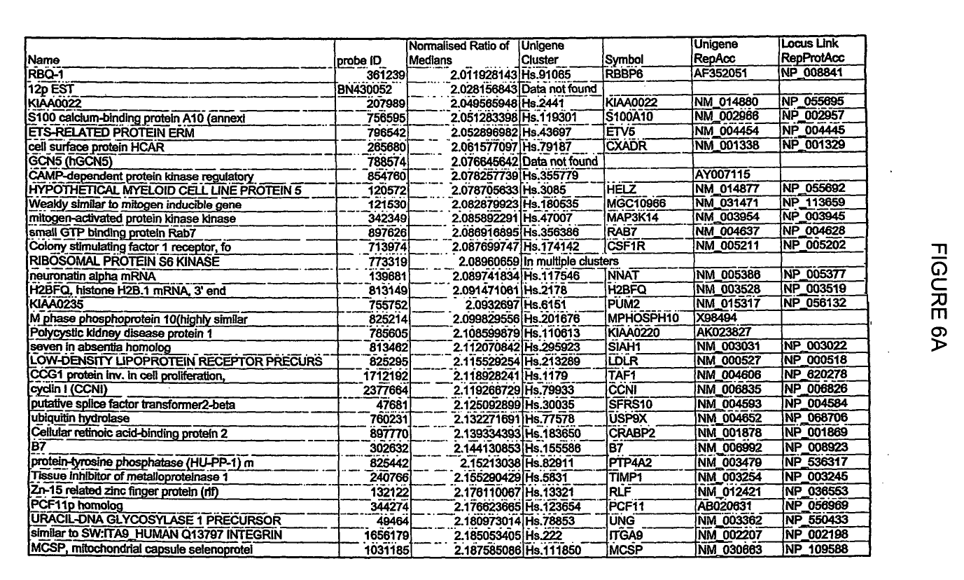

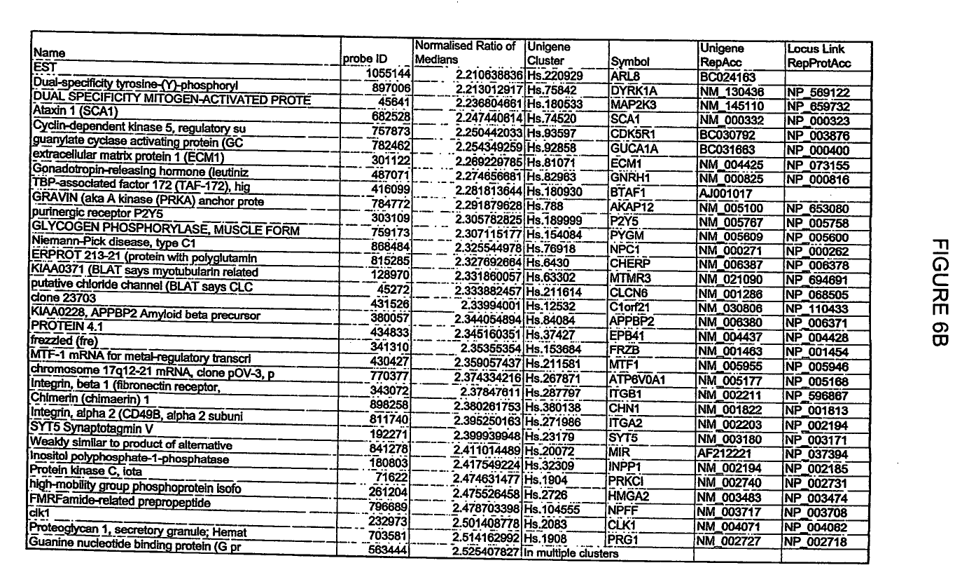

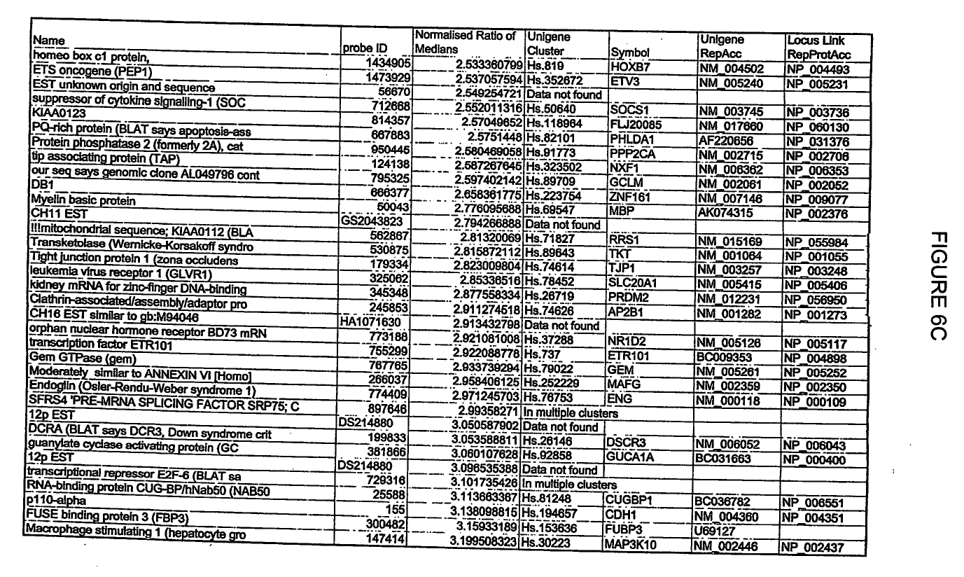

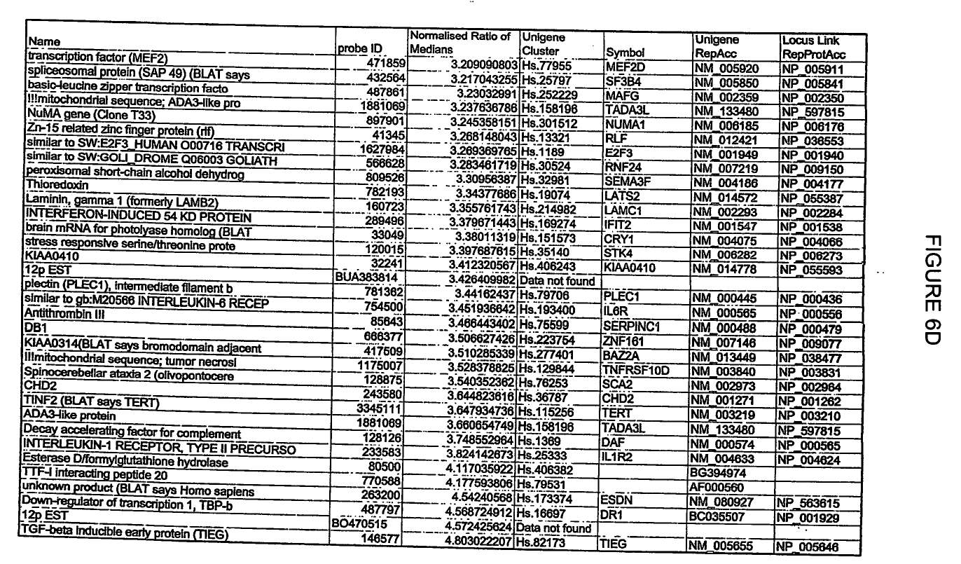

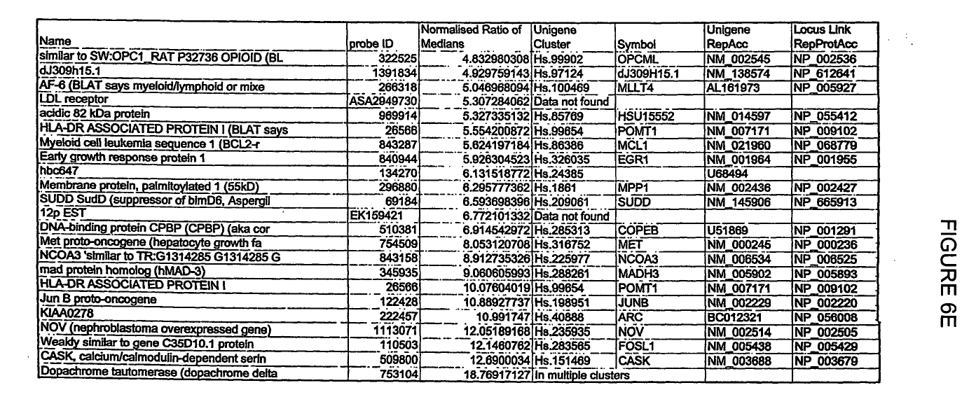

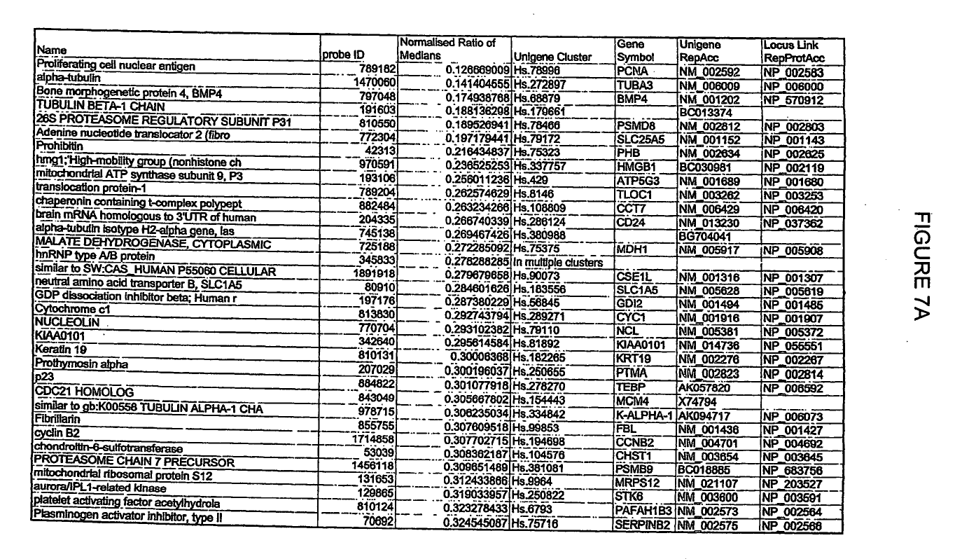

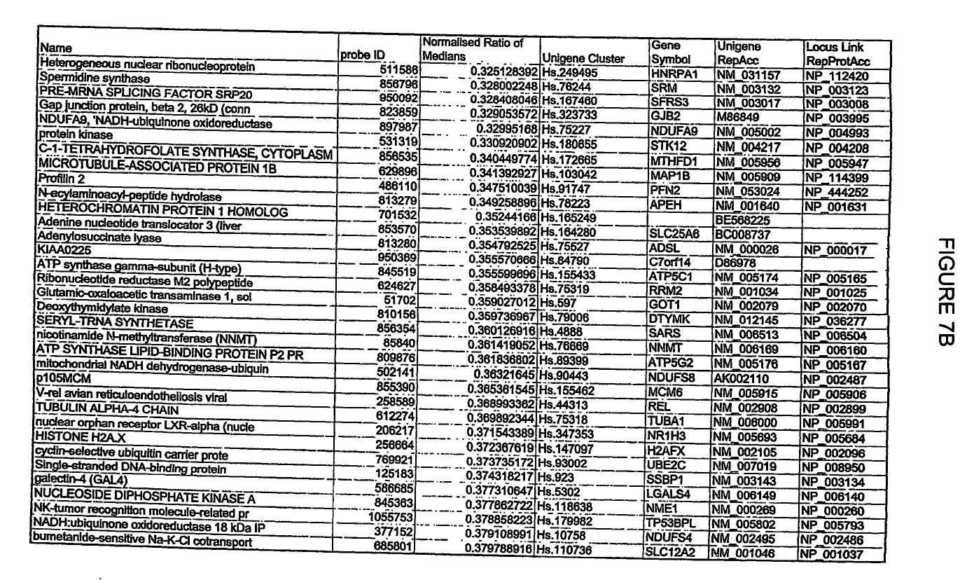

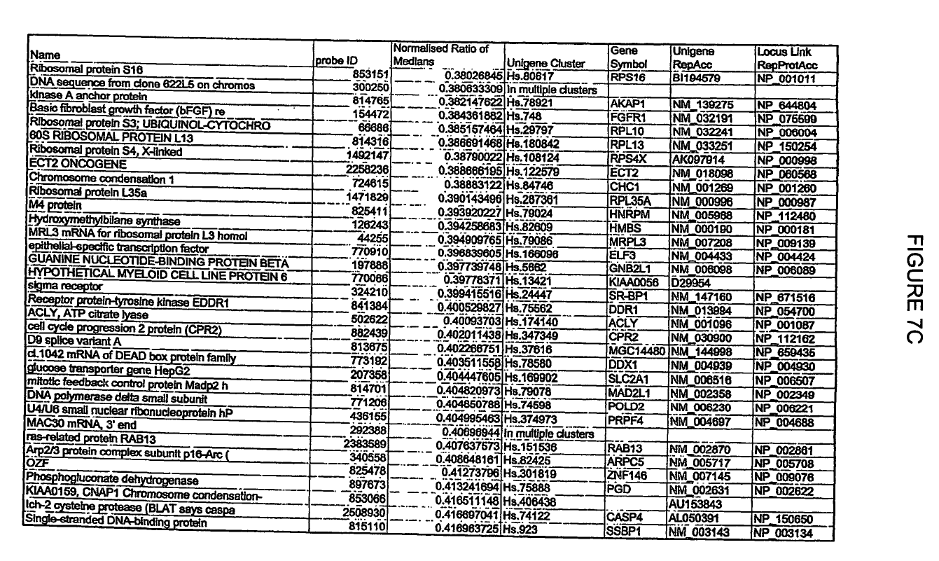

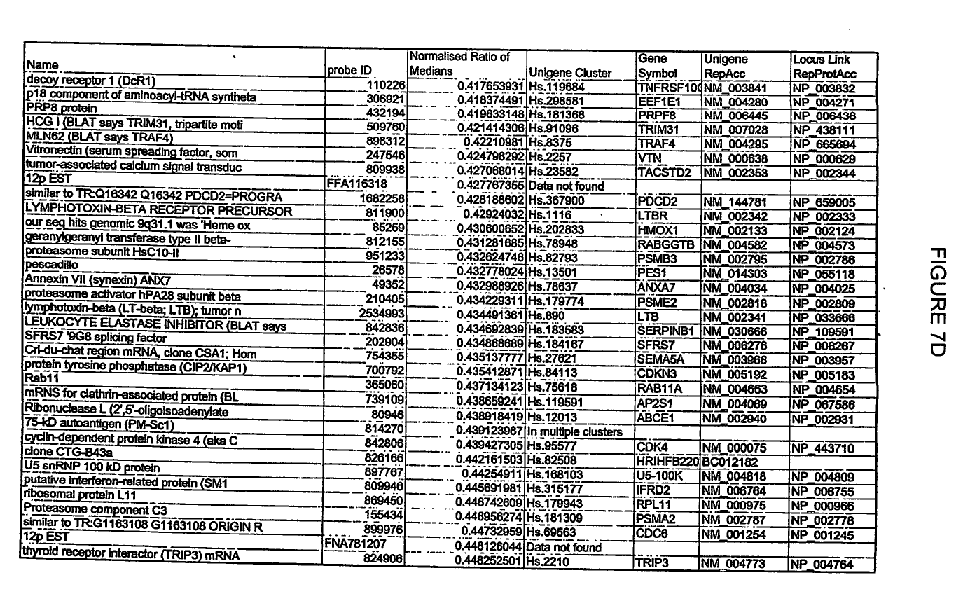

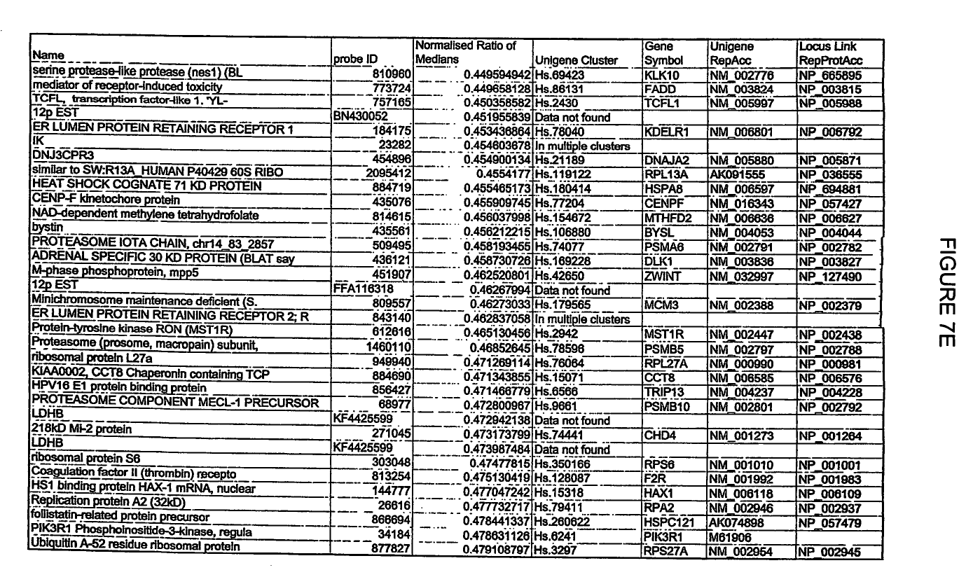

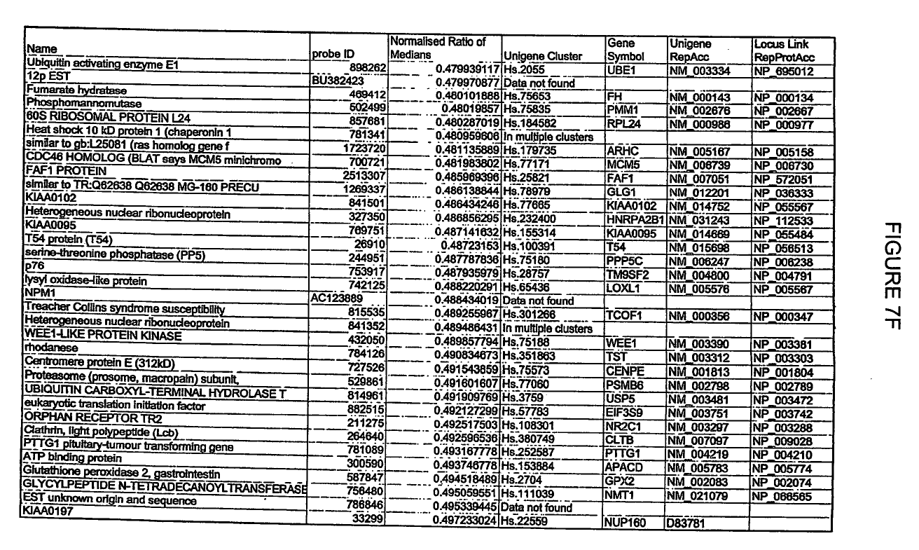

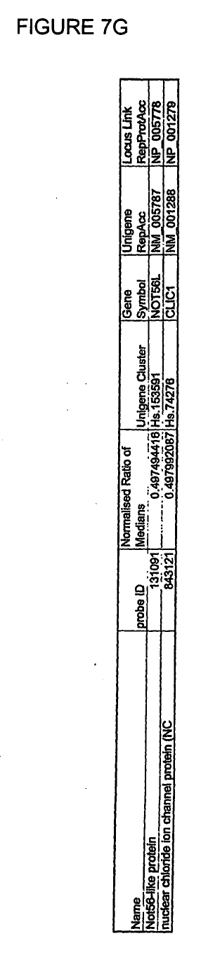

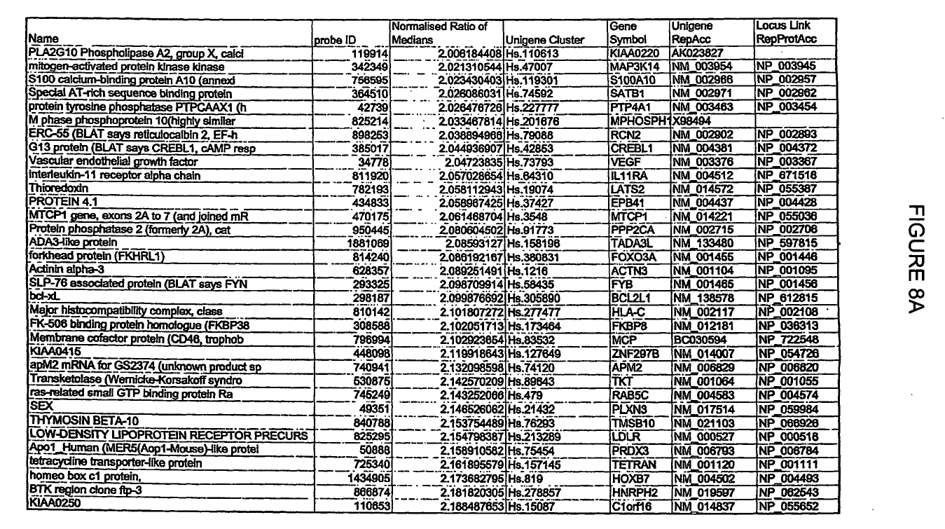

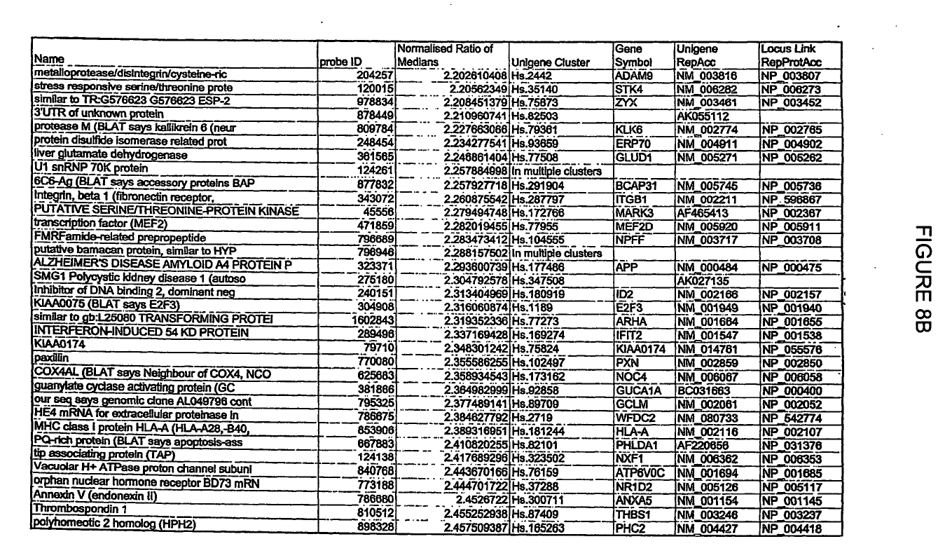

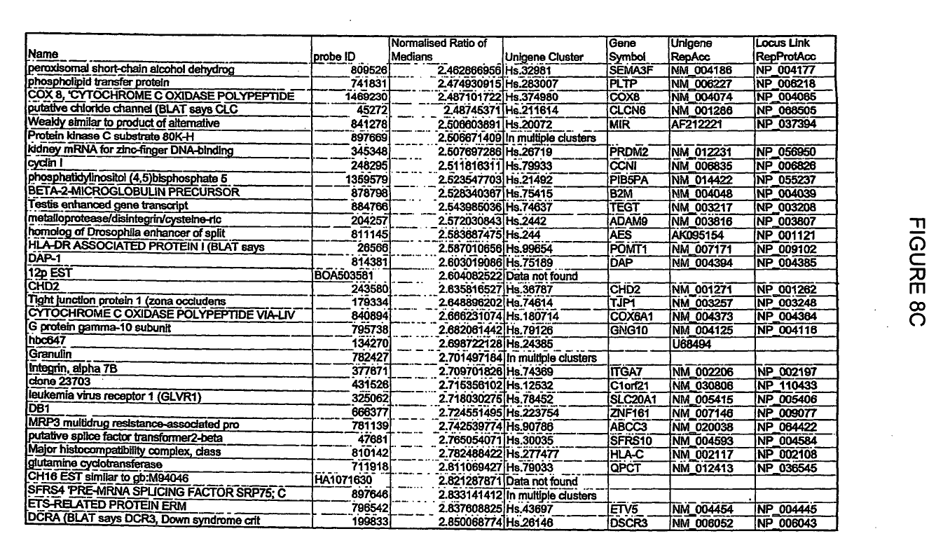

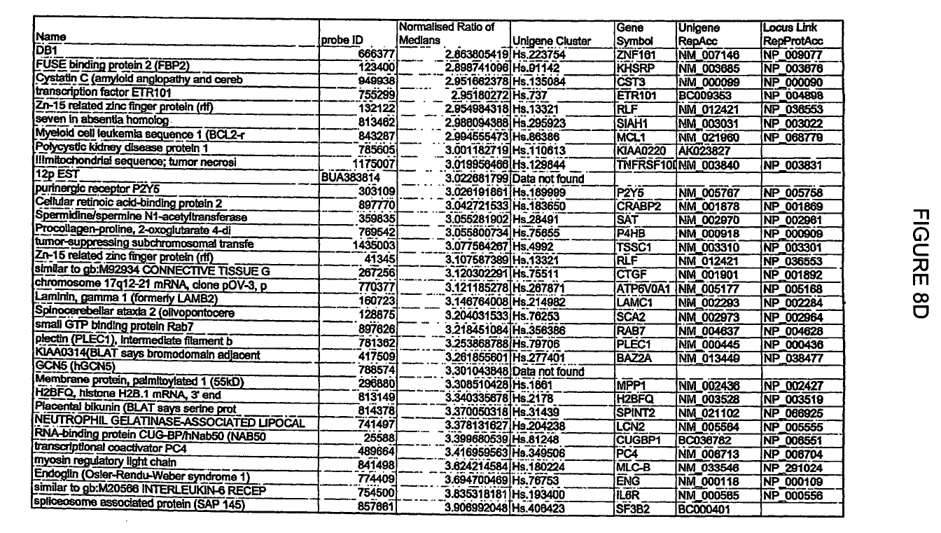

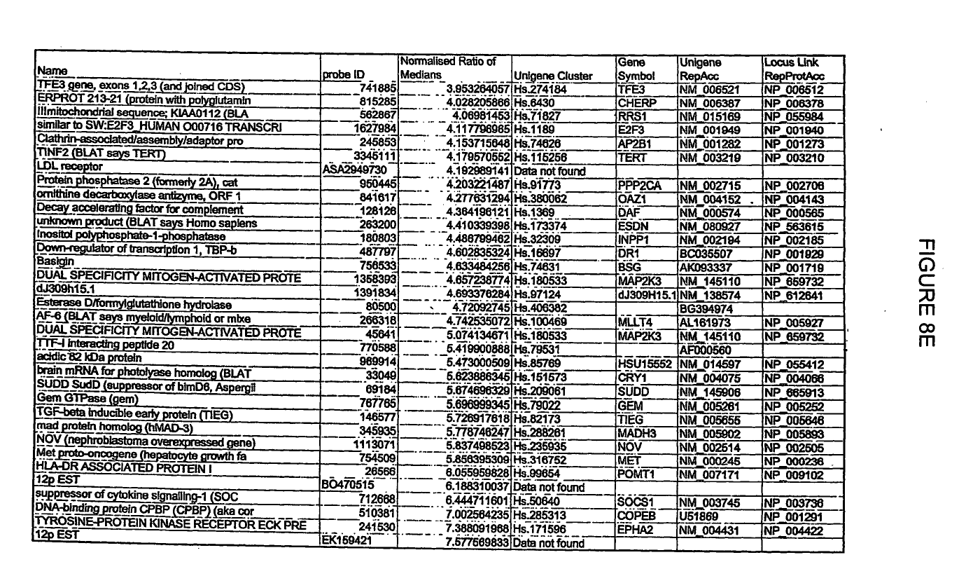

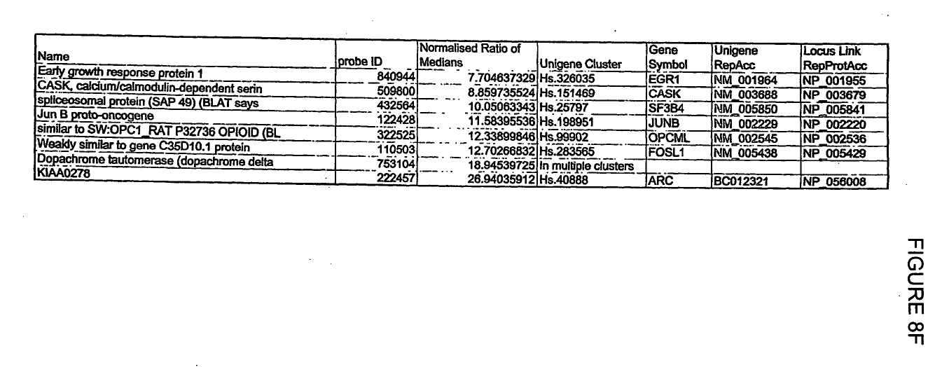

In the context of the present invention, array technology can be used, for example, in the analysis of the expression of one or more of the protein markers of roscovitine activity identified herein. In one embodiment, array technology may be used to assay the effect of a candidate compound on a number of the markers of roscovitine activity identified herein simultaneously. Accordingly, another aspect of the present invention is to provide microarrays that include at least one, at least two or at least several of the nucleic acids identified in any of FIGS. 1 through 8, or fragments thereof, or protein or antibody arrays.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array. In the context of the present invention, a “library” may include a sample of markers of roscovitine activity as identified herein.

The samples (e.g., members of a library) are generally fixed or immobilised onto a solid phase, preferably a solid substrate, to limit diffusion and admixing of the samples. In a preferred embodiment, libraries of DNA binding ligands may be prepared. In particular, the libraries may be immobilised to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilised on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica based substrates. Thus, the samples are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14(11):26.

Techniques for producing immobilised libraries of DNA molecules have been described in the art. Generally, most prior art methods described how to synthesise single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

Arrays of peptides (or peptidomimetics) may also be synthesised on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854; WO 90/15070 and WO 92/10092; Fodor et al., 1991, Science 251:767; Dower and Fodor, 1991, Ann. Rep. Med. Chem. 26:271.

To aid detection, targets and probes may be labelled with any readily detectable reporter, for example, a fluorescent, bioluminescent, phosphorescent, radioactive, etc reporter. Such reporters, their detection, coupling to targets/probes, etc are discussed elsewhere in this document. Labelling of probes and targets is also disclosed in Shalon et al., 1996, Genome Res 6(7):639-45.

Specific examples of DNA arrays include the following:

Format I: probe cDNA (˜500-˜5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 17:217-218).

Format II: an array of oligonucleotide (˜20-˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such a DNA chip is sold by Affymetrix, Inc., under the GeneChip® trademark.

Examples of some commercially available microarray formats are set out, for example, in Marshall and Hodgson, 1998, Nature Biotechnology 16(1):27-31.

Data analysis is also an important part of an experiment involving arrays. The raw data from a microarray experiment typically are images, which need to be transformed into gene expression matrices—tables where rows represent for example genes, columns represent for example various samples such as tissues or experimental conditions, and numbers in each cell for example characterize the expression level of the particular gene in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J, 2000, FEBS Lett 480(1):17-24.

As disclosed above, proteins, polypeptides, etc may also be immobilised in arrays. For example, antibodies have been used in microarray analysis of the proteome using protein chips (Borrebaeck Calif., 2000, Immunol Today 21(8):379-82). Polypeptide arrays are reviewed in, for example, MacBeath and Schreiber, 2000, Science, 289(5485):1760-1763.

Diagnostics and Prognostics

The invention also includes use of the markers of roscovitine activity, antibodies to those proteins, and compositions comprising those proteins and/or their antibodies in diagnosis or prognosis of diseases characterized by proliferative activity, particularly in individuals being treated with roscovitine. As used herein, the term “prognostic method” means a method that enables a prediction regarding the progression of a disease of a human or animal diagnosed with the disease, in particular, cancer. In particular, cancers of interest with respect to roscovitine treatment include breast, lung, gastric, head and neck, colorectal, renal, pancreatic, uterine, hepatic, bladder, endometrial and prostate cancers and leukemias.

In one embodiment, prognostics may include detecting the expression of markers whose expression correlates with roscovitine sensitivity or resistance in a method of predicting the response of a patient to treatment.

The term “diagnostic method” as used herein means a method that enables a determination of the presence or type of cancer in or on a human or animal. Suitably the marker allows success of roscovitine treatment to be assessed. As discussed above, suitable diagnostics include probes directed to any of the genes as identified herein such as, for example, QPCR primers, FISH probes and so forth.

The present invention will now be described with reference to the following examples.

### EXAMPLES

Methods

Cell Culture

HT29 colon cancer cells were seeded into T175 flasks at 3×10⁶ cells per flask and allowed to attach for 48 h. Cells were then treated with 50 μM CYC202 for either 4, 12, 24 or 48 h prior to harvesting by trypsinisation. A cell pellet was made for protein analysis and RNA analysis.

Western Blotting

To harvest cells, the medium was removed and cells were incubated with 5 ml trypsin for 5 min at 37° C. to detach them from the plastic. The cells were then pelleted, washed in ice cold PBS and resuspended in ice cold lysis buffer containing 50 mM HEPES pH7.4, 250 mM NaCl, 0.1% NP40, 1 mM DTT, 1 mM EDTA, 1 mM NaF, 10 mM β-glycerophosphate, 0.1 mM sodium orthovanadate and 1 complete protease inhibitor cocktail tablet (Roche, East Sussex, UK) per 10 ml of lysis buffer for 30 minutes on ice. Lysates were centrifuged at approx. 18, 000×g for 10 minutes at 4° C. to remove cellular debris. The supernatant was stored at −80° C. prior to use. The protein concentration of lysates was determined using the BCA protein assay (Pierce, Rockford, USA). Proteins were separated by SDS-PAGE using Novex precast tris-glycine gels (Invitrogen, Groningen, The Netherlands) and transferred to Immobilon-P membranes (Millipore, Bedford, USA). Membranes were blocked for 1 hour in TBS™ 50 mM Tris pH7.5, 150 mM NaCl, 0.1% Tween 20 (Sigma, Dorset, UK) and 3% milk. Immunoblotting with primary antibodies diluted in TBS™ was performed at 4° C. overnight, followed by a 1 hour incubation with HRP-conjugated secondary antibodies at room temperature. Membranes were washed with ECL reagents and exposed to Hyperfilm (Amersham Pharmacia Biotech, Buckinghamshire, UK). Antibodies used were: phospho-RB Ser780 1:5000, phospho-ERK1/2 1:1000, c-JUN 1:200 (Cell Signalling Technologies, Beverly, USA), total RB SC-50 1:2000, cyclin B2 SC-5233 1:100, EGR-1 1:200 SC-189 (Santa Cruz Biotechnology, Santa Cruz, USA), total ERK2 1:10000 (kindly provided by Prof. Chris Marshall, Institute of Cancer Research, London, UK), phospho-RB Ser608 1:2000 (Dr. Sibylle Mittnacht, Institute of Cancer Research, London, UK), phospho-RB Thr821 1:1000 (Biosource, Nivelles, Belgium), non-phosphorylated RB 1:500, Aurora 1 1:250, MCL-1 2 μg/ml (BD Biosciences, Oxford, UK), PLK-1 2 μg/ml Zymed, San Francisco, Calif.), GAPDH 1:5000 (Chemicon, Temecula, Calif.) goat anti-rabbit and goat anti-mouse HRP-conjugated secondary antibodies 1:5000 (BioRad, Hercules, USA), rabbit anti-sheep HRP-conjugated secondary antibody 1:2000 (Upstate Biotechnology, Lake Placid, USA).

Microarray Analysis

Total RNA was extracted from cell pellets using TriZol (Life Technologies) and mRNA was purified using the Qiagen Oligotex system. mRNA from control and treated cells was labelled with either Cy3 or Cy5 (NEN or Amersham) fluorescent dCTPs respectively, creating cDNA probes. cDNA microarray slides are made and used as described in Eisen, MB and Brown PO. (1999) DNA Arrays for Analysis of Gene Expression. Methods in Enzymology 303:179-205.

The cDNA probes were then hybridised to the cDNA microarray slides overnight and then washed and scanned using an Axon Labs GenePix 4000B scanner. The slides were verified using GenePix software and normalised prior to analysis in GeneSpring.

Results

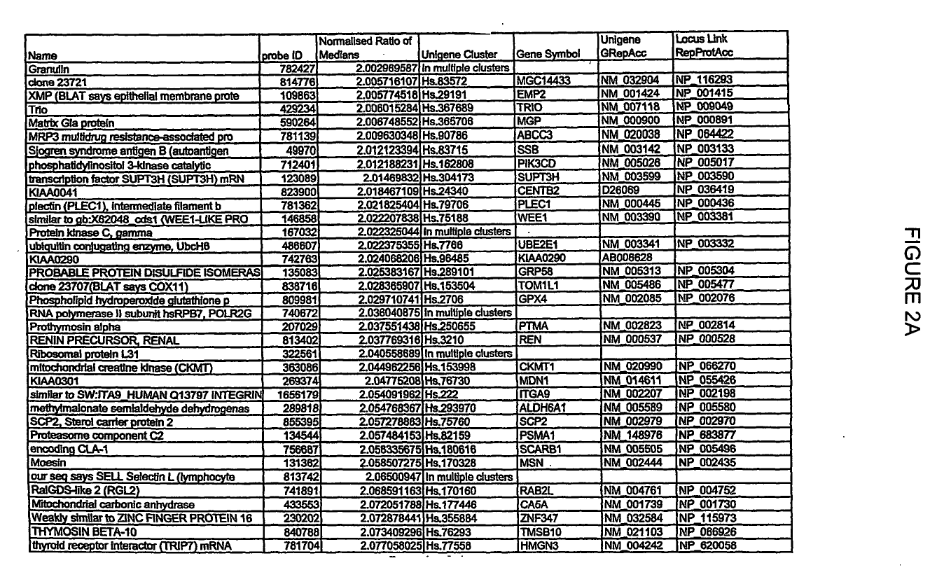

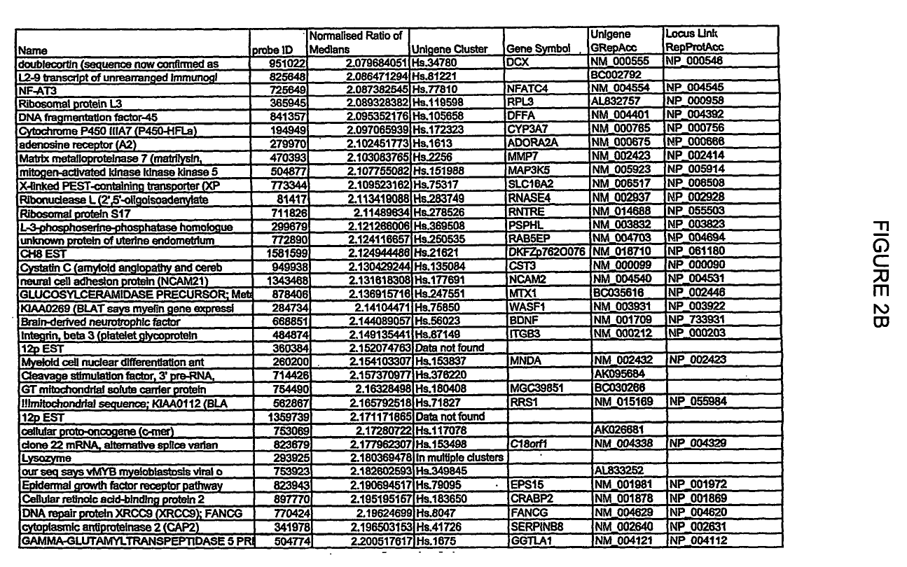

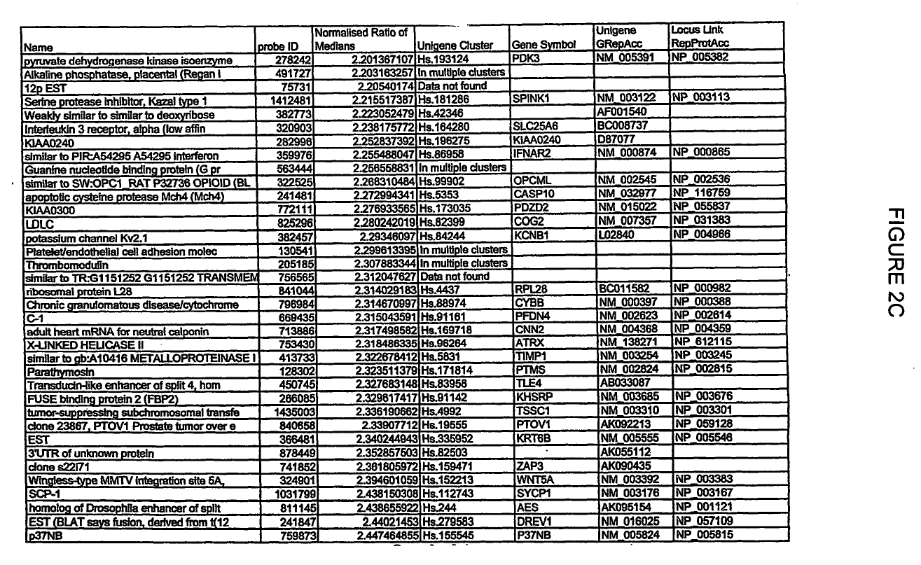

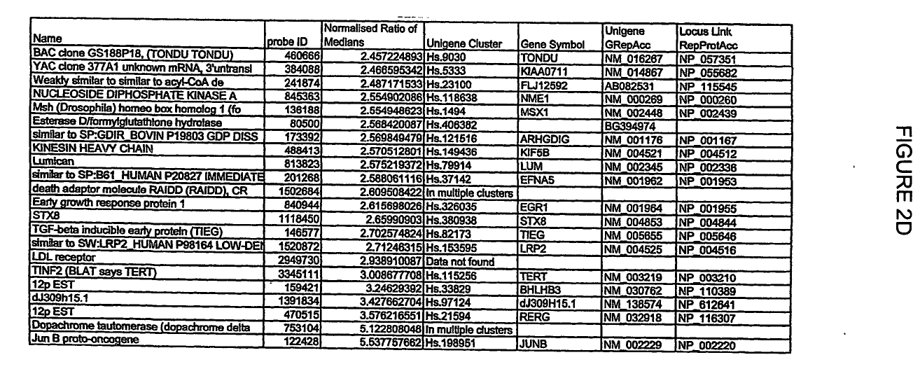

FIGS. 1 through 8 represent the mRNA expression profiles of HT29 cells treated with 50 μM CYC202 for 4, 12, 24 and 48 h respectively when compared to asynchronous control cells. A 2 fold cut-off was used to assign significance to a change in mRNA expression. Therefore, any mRNA with a normalised ratio of medians less than 0.5 (FIGS. 1, 3, 5 and 7) or greater than 2 (FIGS. 2, 4, 6 and 8) is deemed significant.

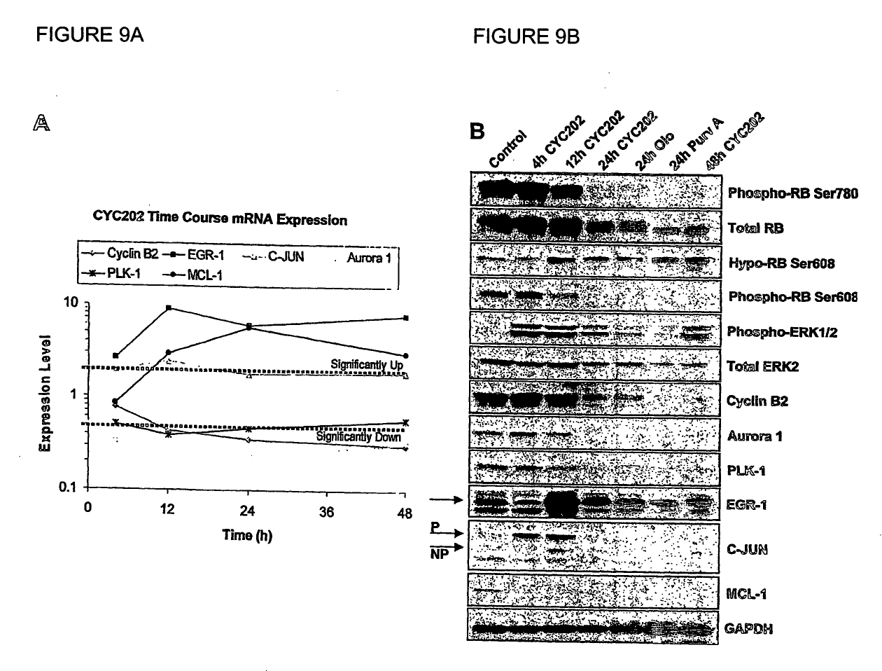

FIG. 9A shows the mRNA expression over the time course of 50 μM CYC202 in HT29 cells of selected genes of interest. These changes were then validated by Western blotting (FIG. 9B). 24 h treatments with olomoucine (174 μM) and purvalanol A (12 μM) were included for comparison.

Relating the microarray data to the Western validation, cyclin B2, aurora 1 and polo-like kinase 1 are all markedly inhibited in agreement with the microarray data. EGR-1 mRNA is induced from as early as 4 h and is maintained above the two fold cut off for the duration of the experiment whereas EGR-1 protein is transiently induced at 12 h after treatment. c-JUN mRNA is induced to significant levels from 4 h and is maintained at that level for the duration of the experiment, c-JUN protein is induced at 4 h and 12 h (and is in the phosphorylated, active form) but is lost after 24 h.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of monitoring the activity of roscovitine comprising:
(i) administering roscovitine to a cell, group of cells, an animal model or human;
(ii) measuring gene expression in samples derived from the treated and the untreated cells, animal or human; and
(iii) detecting an increase or a decrease in gene expression of at least one of the genes identified in FIGS. 1 through 8 in the treated sample as compared to the untreated sample as an indication of roscovitine activity.

2. A method according to claim 1 wherein roscovitine is administered to a mammal.

3. A method according to claim 1, wherein roscovitine is administered to a human.

4. A method according to claim 2, wherein roscovitine is administered to a human.

5. A method according to claim, wherein the group of cells is a cell culture.

6. A method according to claim 5, wherein the cells are selected from PBMC, HT29, and A549 cells.

7. A method according to any one of claims 1 to 6, wherein the presence of at least one of the genes identified in FIGS. 1 through 8 is detected in tumor cells or lymphocytes.

8. A method according to any one of claims 1 to 6, wherein the level of at least one of the genes identified in FIGS. 1, 3, 5 and 7 is less than that detected prior to administration of roscovitine.

9. A method according to any one of claims 1 to 6, wherein the level of at least one of the genes identified in FIGS. 2, 4, 6 and 8 is greater than that detected prior to administration of roscovitine.

10. A method of assessing suitable dose levels of roscovitine comprising monitoring the degree and rate of expression of at least one of the genes identified in FIGS. 1 through 8 after administration of roscovitine to a cell, group of cells, animal model or human.

11. A method of identifying a candidate drug having roscovitine-like activity comprising administering said candidate drug to cell, group of cells, animal model or human, and monitoring the presence or absence of at least one of the genes as identified in FIGS. 1 through 8.

12. A method according to any one of claims 1-6, 10 and 11, wherein roscovitine is R-roscovitine.

13. A method of monitoring the activity of roscovitine comprising:
(i) administering roscovitine to a cell, group of cells, an animal model or human;
(ii) measuring the expression of at least one gene derived from the treated and the untreated cells, animal or human, wherein said at least one gene is selected from the group consisting of the genes listed in FIGS. 1 through 8; and
(iii) detecting an increase or a decrease in gene expression of at least one of the genes identified in FIGS. 1 through 8 in the treated sample.

14. The method according to claim 13, wherein the presence of said least one of the genes is monitored after the administration of roscovitine to said cell, group of cells, an animal model or human.

15. The method according to claim 13 or 14, wherein roscovitine is R-roscovitine.

16. A kit for assessing the activity of roscovitine comprising an antibody for each of at least one of the genes as identified in FIGS. 1 through 8.

17. A kit for assessing the activity of roscovitine comprising nucleic acid probes which specifically hybridizes to at least one of said genes identified in FIGS. 1 through 8.

18. A method of monitoring the activity of a CDKI comprising:
(i) administering said CDKI to a cell, group of cells, an animal model or human; and
(ii) measuring the expression of at least one of the genes identified in any of FIGS. 1 thorugh 8 in samples derived from the treated and the untreated cells, animal or human; and
(iii) detecting an increase or a decrease in expression of at least one of the genes identified in any of FIGS. 1 through 8 in the treated sample as compared to the untreated sample as an indication of CDKI activity.

19. The method of claim 13 or claim 18, wherein said increase or decrease in gene expression is detected in a method comprising antibodies which specifically bind at least one of the genes identified in FIGS. 1 through 8.

20. The method of claim 13 or claim 18, wherein said increase or decrease in gene expression is detected in a method comprising a nucleic acid probe which is specific for at least one of the genes identified in FIGS. 1 through 8.
